(19) United States
(12) Patent Application Publication
(10) Pub. No.: US 2024/0124563 A1
(43) Pub. Date: Apr. 18, 2024

(54) Anti-Human MSLN Antibody And Application Thereof

(71) Applicant: NANJING ZAIMING PHARMACEUTICAL CO., LTD., Jiangsu (CN)

(72) Inventors: Cuiqing YANG, Shanghai (CN); Xiaohui SHAO, Shanghai (CN); Zhuoxiao CAO, Shanghai (CN); Renhong TANG, Shanghai (CN); Jinsheng REN, Jiangsu (CN)

(73) Assignee: NANJING ZAIMING PHARMACEUTICAL CO., LTD., Jiangsu (CN)

(21) Appl. No.: 18/265,591

(22) PCT Filed: Dec. 8, 2021
(86) PCT No.: PCT/CN2021/136419

§ 371 Date: Jun. 6, 2023

(30) Foreign Application Priority Data
Dec. 9, 2020 (CN) 202011424591.7

(51) Int. Cl. C07K 16/18; A61K 39/395; A61K 45/06

(52) U.S. Cl. CPC C07K 16/18; A61K 39/3955; A61K 45/06; C07K 2317/31; C07K 2317/33; C07K 2317/35; C07K 2317/569; C07K 2317/92

(57) ABSTRACT

The present invention relates to a single-domain antibody against MSLN (mesothelin) and a preparation method therefor and an application thereof. The MSLN antibody has high affinity for MSLN, and therefore can be applied to the preparation of a medicament for treating tumors, etc.

The present invention claims the priority to the Chinese Patent Application No. 202011424591.7 entitled “ANTI-HUMAN MSLN ANTIBODY AND APPLICATION THEREOF” filed with National Intellectual Property Administration, PRC on Dec. 9, 2020, which is incorporated into the present invention by reference in its entirety.

### TECHNICAL FIELD

The present invention belongs to the fields of bioengineering and biomedicines, and relates to an anti-human MSLN antibody, a nucleic acid for encoding the antibody, an antibody preparation method, a pharmaceutical composition containing the antibody, and related use of the pharmaceutical composition in treating tumors.

### BACKGROUND

Mesothelin (MSLN) is a differentiation antigen present on normal mesothelial cells, and can be expressed in the mesothelial cells of the normal pleurae, pericardia and peritonea. Although its expression is limited in normal tissues, MSLN has been found to be expressed in 90% of epithelioid malignant pleural mesothelioma cells, 69% of lung adenocarcinoma cells, 60% of breast cancer cells, 46% of esophageal cancer cells, pancreatic tumor cells, and ovarian cancer cells (Morello A et al., Cancer Discov. 2016; 6(2):133-146; Baldo P et al., Onco Targets Ther. 2017; 10:5337-5353; Argani P et al., Clin Cancer Res. 2001; 7(12):3862-3868; Hassan R et al., Clin Cancer Res. 2004; 10(12 Pt 1):3937-3942). Therefore, MSLN is likely to be an important target for cancer therapy. The MSLN gene which is located on chromosome 16 p13.3 has a total length of 8 kb, with a cDNA length of 2138 bp, has a 1884-bp open reading frame, contains 17 exons, and encodes 628 amino acids. The MSLN gene encodes a precursor protein of 71 kDa. The MSLN precursor protein is anchored to the cell membrane by the glycophosphatidylinositol (GPI), and can be hydrolyzed by furin into two portions: an N-terminus soluble protein with a molecular weight of 31 kDa (known as megakaryocyte-potentiating factor (MPF)) and a cell surface glycoprotein with a molecular weight of 40 kDa, which is the mature MSLN (Chang K et al., Proc Natl Acad Sci USA. 1996; 93(1):136-140; Manzanares M Á et al., Hepatol Commun. 2017; 2(2):155-172).

The biological function of mesothelin has not yet been fully elucidated. Researchers studied mice with the MSLN gene knocked out and found that the mice showed no abnormalities in development, reproduction and blood cell count, indicating that it did not affect the normal growth and development of the mice. (Bera T K et al., Mol Cell biol. 2000; 20 (8):2902-2906).

The abnormal expression of MSLN plays an important role in the proliferation, differentiation, adhesion and drug resistance of tumor cells. The overexpression of MSLN can activate NF-κB (nuclear factor kappa-light-chain-enhancer of activated B cells), MAPK (mitogen-activated protein kinase) and PI3K (phosphoinositide 3-kinases) signaling pathways to induce cell apoptosis, or promote cell proliferation, migration and metastasis by inducing the activation and expression of MMP7 (matrix metalloproteinase-7) and MMP9 (matrix metalloproteinase-9). Studies have shown that MSLN can block taxol-induced apoptosis of tumor cells and increase the tolerance of cancer cells to drugs by simultaneously activating PI3K/AKT (protein kinase B, PKB) and MAPK/ERK (extracellular regulated protein kinases) signaling pathways (Bharadwaj U et al., Mol cancer. 2011; 10:106; Cheng W F et al., Br J cancer. 2009; 100(7):1144-1153).

Traditional monoclonal antibodies have high molecular weight, poor tissue permeability, and limited therapeutic effect; murine monoclonal antibodies have high immunogenicity, and the affinity maturation of engineered chimeric antibodies and humanized antibodies is more challenging; the research, development and popularization of fully human monoclonal antibodies are also limited by factors such as high preparation cost, long development cycle, low yield, etc.

In 1993, Belgian scientists discovered for the first time that a class of heavy-chain antibodies lacking light chains exist in camel blood, which only contains a heavy chain variable region and two conventional CH2 and CH3 regions, but has good structural stability and antigen-binding activity, and a single-domain antibody which only consists of the heavy chain variable region, also called VHH (variable domain of heavy chain of heavy-chain antibody) or nanobody, can be obtained by cloning the variable region. The molecular weight of the single-domain antibody is only 1/10 of that of a common antibody, and the single-domain antibody is the smallest functional antigen-binding fragment, which has the advantages of flexible chemical properties, easy expression, good solubility, strong permeability, weak immunogenicity, simple humanization, easy conjugation with other molecules, etc., making up for the defects of traditional antibodies and increasing the diversity of drug development.

### SUMMARY

The present invention provides an anti-human MSLN antibody, a nucleic acid for encoding the antibody, an antibody preparation method, a pharmaceutical composition containing the antibody, and related use of the pharmaceutical composition in treating tumors.

In a first aspect, the present invention provides an antibody or antigen-binding fragment that may specifically bind to MSLN, comprising: a CDR1, a CDR2, and a CDR3, wherein the CDR1, the CDR2, and the CDR3 have a sequence combination selected from any one of the following sequence combinations or a sequence combination with 1, 2, 3, or more amino acid insertions, deletions and/or substitutions compared with the sequence combinations and the CDR1, the CDR2, and the CDR3 are encoded according to the universal analysis method of KABAT, Chothia, or IMGT:
(1) the CDR1 may be selected from SEQ ID NOs: 35, 38, 41, 44, 47, 50, 53, 56, 59, 62, 65, 68, 71, 74, 77, 80, 83, 86, 89, 92, 95, 98, 101, 104, 107, 110, 113, 116, 119, 122, 125, 128, 131, 134, 137, 140;
(2) the CDR2 may be selected from SEQ ID NOs: 36, 39, 42, 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141;
(3) the CDR3 may be selected from SEQ ID NOs: 37, 40, 43, 46, 49, 52, 55, 58, 61, 64, 67, 70, 73, 76, 79, 82, 85, 88, 91, 94, 97, 100, 103, 106, 109, 112, 115, 118, 121, 124, 127, 130, 133, 136, 139, 142.

In some embodiments, preferably, the antibody or the antigen-binding fragment comprises a CDR1, a CDR2 and a CDR3, selected from a VHH domain set forth in any one of SEQ ID NOs: 23 to 34 respectively, and according to the KABAT, Chothia, or IMGT numbering scheme, the CDR1, the CDR2 and the CDR3 are selected from the following:
(1) the CDR1 is selected from SEQ ID NOs: 35, 71, 107, the CDR2 is selected from SEQ ID NOs: 36, 72, 108, and the CDR3 is selected from SEQ ID NOs: 37, 73, 109;
(2) the CDR1 is selected from SEQ ID NOs: 38, 74, 110, the CDR2 is selected from SEQ ID NOs: 39, 75, 111, and the CDR3 is selected from SEQ ID NOs: 40, 76, 112;
(3) the CDR1 is selected from SEQ ID NOs: 41, 77, 113, the CDR2 is selected from SEQ ID NOs: 42, 78, 114, and the CDR3 is selected from SEQ ID NOs: 43, 79, 115;
(4) the CDR1 is selected from SEQ ID NOs: 44, 80, 116, the CDR2 is selected from SEQ ID NOs: 45, 81, 117, and the CDR3 is selected from SEQ ID NOs: 46, 82, 118;
(5) the CDR1 is selected from SEQ ID NOs: 47, 83, 119, the CDR2 is selected from SEQ ID NOs: 48, 84, 120, and the CDR3 is selected from SEQ ID NOs: 49, 85, 121;
(6) the CDR1 is selected from SEQ ID NOs: 50, 86, 122, the CDR2 is selected from SEQ ID NOs: 51, 87, 123, and the CDR3 is selected from SEQ ID NOs: 52, 88, 124;
(7) the CDR1 is selected from SEQ ID NOs: 53, 89, 125, the CDR2 is selected from SEQ ID NOs: 54, 90, 126, and the CDR3 is selected from SEQ ID NOs: 55, 91, 127;
(8) the CDR1 is selected from SEQ ID NOs: 56, 92, 128, the CDR2 is selected from SEQ ID NOs: 57, 93, 129, and the CDR3 is selected from SEQ ID NOs: 58, 94, 130;
(9) the CDR1 is selected from SEQ ID NOs: 59, 95, 131, the CDR2 is selected from SEQ ID NOs: 60, 96, 132, and the CDR3 is selected from SEQ ID NOs: 61, 97, 133;
(10) the CDR1 is selected from SEQ ID NOs: 62, 98, 134, the CDR2 is selected from SEQ ID NOs: 63, 99, 135, and the CDR3 is selected from SEQ ID NOs: 64, 100, 136;
(11) the CDR1 is selected from SEQ ID NOs: 65, 101, 137, the CDR2 is selected from SEQ ID NOs: 66, 102, 138, and the CDR3 is selected from SEQ ID NOs: 67, 103, 139;
(12) the CDR1 is selected from SEQ ID NOs: 68, 104, 140, the CDR2 is selected from SEQ ID NOs: 69, 105, 141, and the CDR3 is selected from SEQ ID NOs: 70, 106, 142; or
(13) the CDR1, the CDR2 and the CDR3 have a sequence combination with 1, 2, 3, or more amino acid insertions, deletions and/or substitutions compared with the aforementioned sequence combinations (1) to (12); and preferably, the substitution is a conservative amino acid substitution.

In some embodiments, preferably, the antibody or the antigen-binding fragment comprises a sequence combination of CDR1, CDR2, and CDR3 selected from SEQ ID NOs: 23 to 34, or comprises sequences having at least 80%, 85%, 90%, 91%, 92%, 93%, 94%, 95%, 96%, 97%, 98%, 99%, or 100% sequence identity to the CDR1, the CDR2, and/or the CDR3 described above.

In some embodiment, preferably, the antibody or the antigen-binding fragment comprises an FR region in a VHH domain set forth in any one of SEQ ID NOs: 23 to 34, wherein optionally, the antibody or antigen-binding fragment comprises a sequence having at least 80%, 85%, 90%, 91%, 92%, 93%, 94%, 95%, 96%, 97%, 98%, 99%, or 100% sequence identity to the FR region in the VHH domain set forth in any one of SEQ ID NOs: 23 to 34; or, optionally, the antibody or the antigen-binding fragment comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 23 to 34; and the mutation may be selected from an insertion, a deletion, and/or a substitution; the substitution is preferably a conservative amino acid substitution.

In some embodiments, preferably, the antibody or the antigen-binding fragment comprises a sequence set forth in any one of SEQ ID NOs: 23 to 34, wherein optionally, the antibody or antigen-binding fragment comprises a sequence having at least 80%, 85%, 90%, 91%, 92%, 93%, 94%, 95%, 96%, 97%, 98%, 99%, or 100% sequence identity to the sequence set forth in any one of SEQ ID NOs: 23 to 34; or, optionally, the antibody or the antigen-binding fragment comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 23 to 34; the mutation may be selected from an insertion, a deletion, and/or a substitution; the substitution is preferably a conservative amino acid substitution.

In some embodiments, preferably, the antibody or the antigen-binding fragment binds to human MSLN with a dissociation constant (KD) not greater than 20 nM.

Further, in some embodiments, the antibody or the antigen-binding fragment comprises or does not comprise an antibody heavy chain constant region; optionally, the antibody heavy chain constant region may be selected from human, alpaca (Vicugna pacos), mouse, rat, rabbit, and sheep; optionally, the antibody heavy chain constant region may be selected from IgG, IgM, IgA, IgE, and IgD, and the IgG may be selected from IgG1, IgG2, IgG3, and IgG4; optionally, the heavy chain constant region may be selected from an Fc region, a CH3 region, a heavy chain constant region without a CH1 fragment, and an intact heavy chain constant region; preferably, the heavy chain constant region is a human Fc region, more preferably having an amino acid sequence set forth in SEQ ID NO: 11; preferably, the antibody or the antigen-binding fragment is a single-domain antibody or a heavy-chain antibody.

Further, in some embodiments, the antibody or the antigen-binding fragment is: (1) a chimeric antibody or a fragment thereof; (2) a humanized antibody or a fragment thereof; or (3) a full human antibody or a fragment thereof.

Further, in some embodiments, the antibody or the antigen-binding fragment is further conjugated to a therapeutic agent or a tracer; preferably, the therapeutic agent is selected from a radioisotope, a cytotoxic agent and an immunomodulator, and the tracer is selected from a radiocontrast medium, a paramagnetic ion, a metal, a fluorescent label, a chemiluminescent label, an ultrasound contrast agent, and a photosensitizer; more preferably, the cytotoxic agent is selected from an alkaloid, methotrexate, doxorubicin, a taxane, and a toxin compound; the toxin compound is preferably DM1, DM4, SN-38, MMAE, MMAF, duocarmycin, calicheamicin, and DX8951.

Further, in some embodiments, the antibody or the antigen-binding fragment is further linked to an additional functional molecule; the additional functional molecule may be selected from one or more of: a signal peptide, a protein tag, and a cytokine; preferably, the cytokine may be selected from IL-2, IL-6, IL-12, IL-15, IL-21, IFN, and TNF-alpha.

In a second aspect, the present invention provides a multispecific antibody, comprising the antibody or the antigen-binding fragment according to the first aspect, wherein preferably, the multispecific antibody further comprises an antibody or an antigen-binding fragment that may specifically bind to an antigen other than MSLN or bind to an epitope of MSLN different from that of the antibody or the antigen-binding fragment according to the first aspect.

In some embodiments, preferably, the antigen other than MSLN may be selected from: CD3, preferably CD3ε; CD16, preferably CD16A; CD32B; PD-1; PD-2; PD-L1; VEGF; NKG2D; CD19; CD20; CD40; CD47; 4-1BB; CD137; EGFR; EGFRvIII; TNF-alpha; CD33; HER2; HER3; HAS; CD5; CD27; EphA2; EpCAM; MUC1; MUC16; CEA; Claudin18.2; folate receptor; Claudin6; WT1; NY-ESO-1; MAGE3; and ASGPR1 or CDH16.

In some embodiments, preferably, the multispecific antibody may be a bispecific antibody, a trispecific antibody, or a tetraspecific antibody, and may be bivalent, tetravalent, or hexavalent.

In a third aspect, the present invention provides a chimeric antigen receptor (CAR), at least comprising an extracellular antigen-binding domain, a transmembrane domain, and an intracellular signaling domain, wherein the extracellular antigen-binding domain comprises the antibody or the antigen-binding fragment according to the first aspect.

In a fourth aspect, the present invention provides an immune effector cell expressing the chimeric antigen receptor according to the third aspect, or comprising a nucleic acid fragment encoding the chimeric antigen receptor according to the third aspect, wherein preferably, the immune effector cell is selected from a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell; preferably, the immune effector cell is an autoimmune effector cell or an allogeneic immune effector cell.

In a fifth aspect, the present invention provides an isolated nucleic acid fragment capable of encoding the antibody or the antigen-binding fragment according to the first aspect, the multispecific antibody according to the second aspect, or the chimeric antigen receptor according to the third aspect described above.

In a sixth aspect, the present invention provides a vector comprising the isolated nucleic acid fragment according to the fifth aspect.

In a seventh aspect, the present invention provides a host cell, comprising the vector according to the six aspect, wherein preferably, the cell is a prokaryotic cell or a eukaryotic cell, such as a bacterium (Escherichia coli), a fungus (yeast), an insect cell, or a mammalian cell (a CHO cell or a 293T cell).

In an eighth aspect, the present invention further provides a method for preparing an antibody or an antigen-binding fragment or a multispecific antibody, comprising: culturing the cell according to the seventh aspect described above; and isolating an antibody or an antigen-binding fragment expressed by the cell or a multispecific antibody expressed by the cell in a suitable condition.

In a ninth aspect, the present invention further provides a method for preparing an immune effector cell, wherein the method comprises introducing a nucleic acid fragment encoding the CAR according to the third aspect into the immune effector cell; and optionally, the method further comprises initiating expression of the CAR according to the third aspect in the immune effector cell.

In a tenth aspect, the present invention further provides a pharmaceutical composition, comprising the antibody or the antigen-binding fragment according to the first aspect, the multispecific antibody according to the second aspect, the immune effector cell according to the fourth aspect, the nucleic acid fragment according to the fifth aspect, the vector according to the sixth aspect, or a product prepared by the method according to the eighth or ninth aspect, wherein optionally, the pharmaceutical composition further comprises a pharmaceutically acceptable carrier, a diluent, or an adjuvant; and optionally, the pharmaceutical composition further comprises an additional antineoplastic agent.

In an eleventh aspect, the present invention further provides a method for preventing and/or treating tumors, comprising: administering to a patient in need thereof an effective amount of the antibody or the antigen-binding fragment according to the first aspect, the multispecific antibody according to the second aspect, the immune effector cell according to the fourth aspect, the nucleic acid fragment according to the fifth aspect, the vector according to the sixth aspect, a product prepared by the method according to the eighth or ninth aspect, or the pharmaceutical composition according to the tenth aspect. The tumor is preferably mesothelioma, lung cancer, breast cancer, esophageal cancer, pancreatic cancer, ovarian cancer, or pleural cancer, more preferably epithelioid malignant pleural mesothelioma or lung adenocarcinoma.

In a twelfth aspect, the present invention provides use of the antibody or the antigen-binding fragment according to the first aspect, the multispecific antibody according to the second aspect, the immune effector cell according to the fourth aspect, the nucleic acid fragment according to the fifth aspect, the vector according to the sixth aspect, a product prepared by the method according to the eighth or ninth aspect, or the pharmaceutical composition according to the tenth aspect in preparing a medicament for preventing and/or treating tumors, wherein the tumor is preferably mesothelioma, lung cancer, breast cancer, esophageal cancer, pancreatic cancer, ovarian cancer, or pleural cancer, more preferably epithelioid malignant pleural mesothelioma or lung adenocarcinoma.

In a thirteenth aspect, the present invention provides a kit, comprising the antibody or the antigen-binding fragment according to the first aspect, the multispecific antibody according to the second aspect, the immune effector cell according to the fourth aspect, the nucleic acid fragment according to the fifth aspect, the vector according to the sixth aspect, or a product prepared by the method according to the eighth or ninth aspect.

In a fourteenth aspect, the present invention provides a method for inhibiting the proliferation or migration of a cell expressing MSLN in vitro, comprising: contacting the cell with the antibody or the antigen-binding fragment according to the first aspect in a condition allowing formation of a complex between the antibody or the antigen-binding fragment according to the first aspect and MSLN.

In a fifteenth aspect, the present invention provides a method for detecting MSLN expression, comprising: contacting a cell with the antibody or the antigen-binding fragment according to the first aspect in a condition allowing formation of a complex between the antibody or the antigen-binding fragment according to the first aspect and MSLN.

### Terms and Definitions

Unless otherwise specified, the terms used herein have the meanings that are commonly understood by those of ordinary skill in the art. For a term explicitly defined herein, the meaning of the term shall be subject to the definition.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

As used herein, the terms “including”, “comprising” and “having” are used interchangeably and are intended to indicate the inclusion of a solution, implying that there may be elements other than those listed in the solution. Meanwhile, it should be understood that the descriptions “including”, “comprising” and “having” as used herein further provide the solution of “consisting of . . . ”.

The term “and/or” used herein includes the meanings of “and”, “or”, and “all or any other combination of elements linked by the term”.

As used herein, the term “optional” or “optionally” means that the event or circumstance subsequently described may, but does not necessarily, occur, and that the description includes instances where the event or circumstance occurs or does not occur. For example, “optionally comprising 1 to 3 antibody heavy chain variable regions” means that the antibody heavy chain variable regions may, but not necessarily, be present, and if present, in an amount of 1, 2 or 3.

As used herein, the term “MSLN” refers to mesothelin (MSLN), which is a differentiation antigen present on normal mesothelial cells, and may be expressed in the mesothelial cells of the normal pleurae, pericardia and peritonea. Although the expression is limited in normal tissues, MSLN has been found to be highly expressed in epithelioid malignant pleural mesothelioma cells, lung adenocarcinoma cells, breast cancer cells, esophageal cancer cells, pancreatic tumor cells, ovarian cancer cells, etc. The term “MSLN” includes MSLN proteins of any human and non-human animal species, and specifically includes human MSLN as well as MSLN of non-human mammals.

As used herein, the term “specific binding” means that an antigen-binding molecule (e.g., an antibody) specifically binds to an antigen and substantially identical antigens, generally with high affinity, but does not bind to unrelated antigens with high affinity. Affinity is generally reflected in an equilibrium dissociation constant (KD), with lower KD indicating higher affinity. In the case of antibodies, high affinity generally means having a KD of about 10-7 M or less, about 10-8 M or less, about 1×10-9 M or less, about 1×10-10 M or less, 1×10-11 M or less, or 1×10-12 M or less. KD is calculated as follows: KD=Kd/Ka, where KD represents the dissociation rate and Ka represents the association rate. The equilibrium dissociation constant KD may be measured using a method well known in the art, such as surface plasmon resonance (e.g., Biacore) or equilibrium dialysis.

As used herein, the term “antibody” (Ab) refers to an immunoglobulin molecule which specifically binds to a target antigen or has immunoreactivity, including polyclonal, monoclonal, genetically engineered and other modified forms of antibodies (including, but not limited to, chimeric antibodies, humanized antibodies, full human antibodies, heteroconjugated antibodies (e.g., bispecific, trispecific and tetraspecific antibodies, diabodies, triabodies, and tetrabodies), and antibody conjugates) and antigen-binding fragments of antibodies (including, for example, Fab ‘, F(ab’)2, Fab, Fv, rIgG, and scFv fragments).

The term “antibody” herein includes a typical “four-chain antibody”, which belongs to an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, and a heavy chain constant region CH3 domain; moreover, when the full-length antibody is of IgE isotype, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are connected to each other and to the light chains through disulfide bonds to form a Y-shaped structure. The heavy chain constant regions of an immunoglobulin differ in their amino acid composition and arrangement, and thus in their antigenicity. Accordingly, “immunoglobulin” herein may be divided into five classes, or isotypes of immunoglobulins, i.e., IgM, IgD, IgG, IgA, and IgE, with their corresponding heavy chains being μ, δ, γ, α, and ε chains, respectively. The Ig of the same class may also be divided into different subclasses according to the differences in amino acid composition of the hinge regions and the number and location of disulfide bonds in the heavy chains; for example, IgG may be divided into IgG1, IgG2, IgG3, and IgG4, and IgA may be divided into IgA1 and IgA2. Light chains are divided into κ or λ chains according to differences in the constant regions. Each of the five classes of Ig may have a κ chain or a λ chain. The term “antibody” herein also includes antibodies that do not comprise a light chain, e.g., heavy-chain antibodies (HCAbs) produced by Camelus dromedarius, Camelus bactrianus, Lama glama, Lama guanicoe, Vicugna pacos, and the like, as well as immunoglobulin new antigen receptors (IgNARs) found in Chondrichthyes such as shark.

As used herein, the term “antigen-binding fragment” refers to one or more fragments of an antibody that retain the ability to specifically bind to a target antigen. The antigen-binding function of an antibody may be performed by fragments of a full-length antibody. An antibody fragment may be a Fab, F(ab′)2, scFv, SMIP, diabody, triabody, affibody, nanobody, aptamer or domain antibody. Examples of binding fragments encompassing the term “antigen-binding fragment” of an antibody include, but are not limited to: (i) an Fab fragment, i.e., a monovalent fragment consisting of VL, VH, CL and CH1 domains; (ii) an F(ab)2 fragment, i.e., a bivalent fragment comprising two Fab fragments linked by a disulfide bond at the hinge region; (iii) an Fd fragment consisting of VH and CH1 domains; (iv) an Fv fragment consisting of VL and VH domains of a single arm of an antibody; (V) a dAb comprising VH and VL domains; (vi) a dAb fragment consisting of a VH domain (Ward et al., Nature 341:544-546, 1989) or VHH; (vii) a dAb consisting of a VH or VL domain; (viii) an isolated complementarity determining region (CDR); (ix) a heavy-chain antibody fragment consisting of VHH, CH2, and CH3; and (x) a combination of two or more isolated CDRs, which may optionally be joined by a synthetic linker. Furthermore, although the two domains (VL and VH) of the Fv fragment are encoded by separate genes, these two domains may be joined using a recombination method through a linker that enables them to be made into a single protein chain in which the VL and VH regions are paired to form a monovalent molecule (known as single chain Fv (scFv); see, e.g., Bird et al., Science 242:423-426, 1988 and Huston et al., Proc. Natl. Acad. Sci. USA 85:5879-5883, 1988). These antibody fragments may be obtained using a conventional technique known to those skilled in the art, and these fragments are screened for use in the same manner as intact antibodies. Antigen-binding fragments may be produced by a recombinant DNA technique or enzymatic or chemical cleavage of intact immunoglobulins or, in some embodiments, by a chemical peptide synthesis procedure known in the art.

As used herein, the term “heavy-chain antibody” refers to an antibody that lacks the light chain of a conventional antibody. The term specifically includes, but is not limited to, homodimeric antibodies comprising a VH antigen-binding domain and CH2 and CH3 constant domains in the absence of a CH1 domain.

As used herein, the term “nanobody” refers to a heavy-chain antibody naturally lacking a light chain present in camelid, and the cloning of its variable region can give a single-domain antibody only consisting of a heavy chain variable region (also known as VHH (variable domain of heavy chain of heavy-chain antibody)), which is the smallest functional antigen-binding fragment.

As used herein, the terms “VHH domain”, “nanobody” and “single-domain antibody” (sdAb) have the same meaning and are used interchangeably, referring to a single-domain antibody consisting of only one heavy chain variable region, constructed by the cloning of a variable region of a heavy-chain antibody, which is the smallest antigen-binding fragment with a complete function. Generally, a single-domain antibody consisting of only one heavy chain variable region is constructed by obtaining a heavy-chain antibody naturally lacking a light chain and a heavy chain constant region 1 (CH1) and then cloning a variable region of an antibody heavy chain.

For further description of “heavy-chain antibodies”, “single-domain antibodies”, “VHH domains” and “nanobodies”, see: Hamers-Casterman et al., Nature. 1993; 363; 446-8; review article (Reviews in Molecular Biotechnology 74: 277-302, 2001) by Muyldermans; and the following patent applications mentioned as general background art: WO 94/04678, WO 95/04079 and WO 96/34103; WO94/25591, WO 99/37681, WO 00/40968, WO 00/43507, WO 00/65057, WO 01/40310, WO 01/44301, EP 1134231 and WO 02/48193; WO97/49805, WO 01/21817, WO 03/035694, WO 03/054016 and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787 and WO 06/122825 and other prior art mentioned in these applications.

As used herein, the term “monoclonal antibody” refers to an antibody derived from a single clone (including any eukaryotic, prokaryotic, or phage clone), and is not limited to the production method of the antibody.

As used herein, the term “multispecific” means having at least two antigen-binding sites, each of which binds to a different epitope of the same antigen or a different epitope of a different antigen. Thus, terms such as “bispecific”, “trispecific”, and “tetraspecific” refer to the number of different epitopes to which an antibody/antigen-binding molecule can bind.

As used herein, the term “valency” refers to the presence of a specified number of binding sites in an antibody/antigen-binding molecule. Thus, the terms “monovalent”, “divalent”, “tetravalent” and “hexavalent” refer to the presence of one binding site, two binding sites, four binding sites, and six binding sites, respectively, in an antibody/antigen-binding molecule.

“Full-length antibody”, “complete antibody” and “intact antibody” are used interchangeably herein and refer to an antibody having a structure substantially similar to that of a natural antibody. “Antibody” herein may be derived from any animal, including but not limited to humans and non-human animals which may be selected from primates, mammals, rodents, and vertebrates, such as Camelidae species, Lama glama, Lama guanicoe, Vicugna pacos, sheep, rabbits, mice, rats, or Chondrichthyes (e.g., shark).

As used herein, the term “chimeric antibody” refers to an antibody having a variable sequence of an immunoglobulin derived from one source organism (e.g., rat, mouse, rabbit, or alpaca) and a constant region of an immunoglobulin derived from a different organism (e.g., human). The method for producing a chimeric antibody is known in the art. See, e.g., Morrison, 1985, Science 229 (4719):1202-7; Oi et al., 1986, Bio Techniques 4:214-221; Gillies et al., 1985 J Immunol Methods 125:191-202; the above is incorporated herein by reference.

As used herein, the term “humanized antibody” refers to a genetically engineered non-human antibody that has an amino acid sequence modified to increase homology to the sequence of a human antibody. Generally, all or part of the CDR regions of a humanized antibody are derived from a non-human antibody (donor antibody), and all or part of the non-CDR regions (e.g., variable region FRs and/or constant regions) are derived from a human immunoglobulin (acceptor antibody). The humanized antibody generally retains or partially retains the desired properties of the donor antibody, including but not limited to, antigen specificity, affinity, reactivity, the ability to increase the activity of immune cells, the ability to enhance an immune response, and the like.

As used herein, the term “full human antibody” refers to an antibody having variable regions in which both the FRs and CDRs are derived from human germline immunoglobulin sequences. Furthermore, if the antibody comprises constant regions, the constant regions are also derived from human germline immunoglobulin sequences. The full human antibody herein may include amino acid residues not encoded by human germline immunoglobulin sequences (e.g., mutations introduced by random or site-specific mutagenesis in vitro or by somatic mutations in vivo). However, “full human antibody” herein does not include antibodies in which CDR sequences derived from the germline of another mammalian species (e.g., mouse) have been grafted onto human framework sequences.

As used herein, the term “variable region” refers to a region of the heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conserved framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen-binding specificity. As used herein, the terms “complementarity determining region” and “CDR” are used interchangeably and generally refer to a hypervariable region (HVR) of a heavy chain variable region (VH) or a light chain variable region (VL), which is also known as the complementarity determining region because it can form precise complementarity to an epitope in a spatial structure, where the heavy chain variable chain CDR may be abbreviated as HCDR and the light chain variable chain CDR may be abbreviated as LCDR. The term “framework region” or “FR region” are used interchangeably, referring to those amino acid residues other than the CDRs in an antibody heavy chain variable region or light chain variable region. In general, a typical antibody variable region consists of 4 FR regions and 3 CDR regions in the following order: FR1-CDR1-FR2-CDR2-FR3-CDR3-FR4 (see Kabat et al., Sequences of Protein of Immunological Interest, National Institute of Health, Bethesda, Md. 1987; which is incorporated herein by reference). For example, herein, CDR1-VH, CDR2-VH and CDR3-VH refer to the first CDR, second CDR and third CDR, respectively, of the heavy chain variable region (VH), which constitute a CDR combination (VHCDR combination) of the heavy chain (or the variable region thereof); CDR1-VL, CDR2-VL and CDR3-VL refer to the first CDR, second CDR and third CDR, respectively, of the light chain variable region (VL), which constitute a CDR combination (VLCDR combination) of the light chain (or the variable region thereof).

For further description of the CDRs, see Kabat et al., J. Biol. Chem., 252:6609-6616 (1977); Kabat et al., United States Department of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45:3832-3839 (2008); Lefranc M. P. et al., Dev. Comp. Immunol., 27: 55-77 (2003); and Honegger and Plückthun, J. Mol. Biol., 309:657-670 (2001). The “CDR” herein may be labeled and defined in a manner known in the art, including but not limited to the Kabat numbering scheme, the Chothia numbering scheme, or the IMGT numbering scheme, using tool websites including but not limited to the AbRSA website (http://cao.lab share.cn/AbRSA/cdrs.php), the abYsis website (www.abysis.org/abysis/sequence_input/key_annotation/key_annotation.cgi), and the IMGT website (http://www.imgt.org/3Dstructure-DB/cgi/DomainGapAlign.cgi#results). The CDR herein includes overlaps and subsets of amino acid residues defined in different ways.

As used herein, the term “Kabat numbering scheme” generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

As used herein, the term “Chothia numbering scheme” generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196:901-917; Chothia et al., (1989) Nature 342:878-883).

As used herein, the term “IMGT numbering scheme” generally refers to a numbering scheme based on the international ImMunoGeneTics information system (IMGT) initiated by Lefranc et al., see Lefranc et al., Dev. Comparat. Immunol. 27:55-77, 2003.

As used herein, the term “heavy chain constant region” refers to the carboxyl-terminus portion of an antibody heavy chain that is not directly involved in the binding of the antibody to an antigen, but exhibits effector functions, such as interaction with an Fc receptor, which has a more conserved amino acid sequence relative to the variable domain of the antibody. The “heavy chain constant region” at least comprises: a CH1 domain, a hinge region, a CH2 domain, a CH3 domain, or a variant or fragment thereof. The “heavy chain constant region” includes a “full-length heavy chain constant region” having a structure substantially similar to that of a natural antibody constant region, and a “heavy chain constant region fragment” including only a portion of the full-length heavy chain constant region. Illustratively, a typical “full-length antibody heavy chain constant region” consists of the CH1 domain-hinge region-CH2 domain-CH3 domain. When the antibody is IgE, it further comprises a CH4 domain; and when the antibody is a heavy-chain antibody, it does not include a CH1 domain. Illustratively, a typical “heavy chain constant region fragment” may be selected from an Fc domain and a CH3 domain.

As used herein, the term “light chain constant region” refers to the carboxyl-terminus portion of an antibody light chain that is not directly involved in the binding of the antibody to an antigen. The light chain constant region may be selected from a constant κ domain and a constant λ domain.

As used herein, the term “Fc region” is used to define the C-terminus region of an antibody heavy chain that contains at least one portion of a constant region. The “Fc region” includes Fc regions of native sequences and variant Fc regions. Illustratively, the human IgG heavy chain Fc region may extend from Cys226 or Pro230 to the carboxyl-terminus of the heavy chain. However, an antibody produced by the host cell may undergo post-translational cleavage, cleaving off one or more (particularly one or two) amino acids from the C-terminus of the heavy chain. Therefore, the antibody which is produced by the host cell through the expression of a particular nucleic acid molecule encoding a full-length heavy chain may comprise the full-length heavy chain, or it may comprise a cleaved variant of the full-length heavy chain. This may be the case when the final two C-terminus amino acids of the heavy chain are glycine (G446) and lysine (K447, numbered according to the Kabat EU index). Therefore, the C-terminus lysine (Lys 447) or the C-terminus glycine (Gly 446) and lysine (Lys 447) of the Fc region may or may not be present.

The IgG Fc region comprises IgG CH2 and IgG CH3 domains, and optionally, may further comprise a complete or partial hinge region, but does not comprise a CH1 domain. The “CH2 domain” of the human IgG Fc region typically extends from an amino acid residue at about position 231 to an amino acid residue at about position 340. In one embodiment, a carbohydrate chain is attached to the CH2 domain. The CH2 domain herein may be a native sequence CH2 domain or a variant CH2 domain. The “CH3 domain” comprises the residue in the Fc region at the C-terminus of the CH2 domain (i.e., from the amino acid residue at about position 341 to the amino acid residue at about position 447 of the IgG). The CH3 region herein may be a native sequence CH3 domain or a variant CH3 domain (e.g., a CH3 domain having a “knob” introduced in one strand and a “hole” correspondingly introduced in the other strand; see U.S. Pat. No. 5,821,333, which is explicitly incorporated herein by reference). As described herein, such variant CH3 domain may be used to promote the heterodimerization of two non-identical antibody heavy chains.

Unless otherwise specified herein, the amino acid residues in the Fc region or the constant region are numbered according to the EU numbering scheme, also known as the EU index, as described in Kabat et al., Sequences of Proteins of Immunological Interest, 5thEd. Public Health Service, National Institutes of Health, Bethesda, MD, 1991.

As used herein, the term “conserved amino acid” generally refers to amino acids that belong to the same class or have similar characteristics (e.g., charge, side chain size, hydrophobicity, hydrophilicity, backbone conformation, and rigidity). Illustratively, the amino acids in each of the following groups are conserved amino acid residues of each other, and substitutions of amino acid residues within the groups are substitutions of conserved amino acids:
(1) acidic amino acids: Asp (D) and Glu (E);
(2) basic amino acids: Lys (K), Arg (R), and His (H);
(3) hydrophilic uncharged amino acids: Ser (S), Thr (T), Asn (N), and Gln (Q);
(4) aliphatic uncharged amino acids: Gly (G), Ala (A), Val (V), Leu (L), and Ile (I);
(5) non-polar uncharged amino acids: Cys (C), Met (M), and Pro (P);
(6) aromatic amino acids: Phe (F), Tyr (Y), and Trp (W).

As used herein, the terms “percent (%) sequence identity” and “percent (%) identity” are used interchangeably, referring to the percentage of identity between amino acid (or nucleotide) residues of a candidate sequence and amino acid (or nucleotide) residues of a reference sequence after aligning the sequences and introducing gaps, if necessary, to achieve maximum percent sequence identity (e.g., gaps may be introduced in one or both of the candidate sequence and the reference sequence for optimal alignment, and non-homologous sequences may be omitted for the purpose of comparison). Alignment may be carried out in a variety of ways well-known to those skilled in the art for the purpose of determining percent sequence identity, for example, using publicly available computer software such as BLAST, ALIGN or Megalign (DNASTAIi) software. Those skilled in the art can determine appropriate parameters for measuring alignment, including any algorithms required to achieve maximum alignment of the full length of the aligned sequences. For example, a reference sequence aligned for comparison with a candidate sequence may show that the candidate sequence exhibits 50% to 100% sequence identity over the full length of the candidate sequence or a selected portion of contiguous amino acid (or nucleotide) residues of the candidate sequence. The length of the candidate sequence aligned for the purpose of comparison may be, e.g., at least 30% (e.g., 30%, 40%, 50%, 60%, 70%, 80%, 90%, or 100%) of the length of the reference sequence. When a position in the candidate sequence is occupied by the same amino acid (or nucleotide) residue at the corresponding position in the reference sequence, then the molecules are identical at that position.

As used herein, the term “chimeric antigen receptor (CAR)” refers to an artificial cell surface receptor engineered to express on an immune effector cell and specifically bind to an antigen, which at least comprises: (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

As used herein, the term “antibody conjugate” refers to a conjugate formed by an antibody molecule chemically bonded to an additional molecule directly or through a linker, e.g., an antibody-drug conjugate (ADC) in which the drug molecule is the additional molecule. The “another molecule” may be selected from a therapeutic agent and a tracer; preferably, the therapeutic agent is selected from a radioisotope, a cytotoxic agent, and an immunomodulator, and the tracer is selected from a radiocontrast medium, a paramagnetic ion, a metal, a fluorescent label, a chemiluminescent label, an ultrasound contrast agent, and a photosensitizer; more preferably, the cytotoxic agent is selected from an alkaloid, methotrexate, doxorubicin, and a taxane; more preferably, the cytotoxic agent is DM1, DM4, SN-38, MMAE, MMAF, duocarmycin, calichemicin, or DX8951.

As used herein, the term “nucleic acid” includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. Herein, the term “nucleic acid molecule” encompasses deoxyribonucleic acid (DNA), including, e.g., complementary DNA (cDNA) and genomic DNA; ribonucleic acid (RNA), in particular in the synthetic form of messenger RNA (mRNA), DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone linkages or chemically modified residues. The nucleic acid molecule also encompasses DNA and RNA molecules suitable for use as vectors for direct expression of the antibody of the present invention in vitro and/or in vivo, e.g., in a host or patient. Such DNA (e.g., cDNA) or RNA (e.g., mRNA) vectors may be unmodified or modified. For example, mRNA may be chemically modified to enhance the stability of the RNA vector and/or the expression of the encoded molecule, so that the mRNA can be injected into a subject to produce antibodies in vivo (see, e.g., Stadler et al., Nature Medicine 2017, published online, Jun. 12, 2017, doi: 10.1038/nm.4356 or EP 2 101 823 B1). An “isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location. As used herein, the term “vector” includes nucleic acid vectors, e.g., DNA vectors (e.g., plasmids), RNA vectors, viruses, or other suitable replicons (e.g., viral vectors). Various vectors have been developed for the delivery of polynucleotides encoding foreign proteins into prokaryotic or eukaryotic cells. The expression vector of the present invention contains polynucleotide sequences as well as additional sequence elements, e.g., for expressing proteins and/or integrating these polynucleotide sequences into the genome of mammalian cells. Some vectors that may be used to express the antibody and antibody fragment of the present invention include plasmids containing regulatory sequences (e.g., promoter and enhancer regions) that direct gene transcription. Other useful vectors for expressing the antibody and antibody fragment contain polynucleotide sequences that enhance the rate of translation of these genes or improve the stability or nuclear export of mRNA produced by gene transcription. These sequence elements include, for example, 5′ and 3′ untranslated regions, internal ribosome entry sites (IRESs), and polyadenylation signal sites, so as to direct the effective transcription of the gene carried on the expression vector. The expression vector of the present invention may also contain a polynucleotide encoding a marker for selecting cells containing such a vector. Examples of suitable markers include genes encoding resistance to antibiotics (e.g., ampicillin, chloramphenicol, kanamycin, or nourseothricin).

The step of transforming host cells with recombinant DNA described in the present invention may be performed using a conventional technique well known to those skilled in the art. The obtained transformants may be cultured by a conventional method, and express the polypeptide encoded by the gene of the present invention. The medium used in culturing may be selected from various conventional media depending on the host cells used. The host cells are cultured under conditions suitable for their growth.

As used herein, the term “pharmaceutical composition” refers to a formulation that exists in a form allowing the biological activity of the active ingredient contained therein to be effective, and does not contain additional ingredients having unacceptable toxicity to a subject to which the pharmaceutical composition is administered.

As used herein, the terms “subject” and “patient” refer to an organism that receives treatment for a particular disease or disorder (e.g., a cancer or an infectious disease) as described herein. Examples of the subject and the patient include mammals, such as human, primate, pig, goat, rabbit, hamster, cat, dog, guinea pig, members of the bovine family (such as cattle, bison, buffalo, elk, yak, etc.), cattle, sheep, horse, bison, etc., that are being treated for a disease or disorder (e.g., a cell proliferative disorder, such as a cancer or an infectious disease).

As used herein, the term “treatment” refers to surgical or therapeutic treatment for the purpose of preventing or slowing (reducing) the progression of an undesirable physiological or pathological change, such as a cell proliferative disorder (such as cancer or infectious disease), in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (whether partial or complete), whether detectable or undetectable. Subjects in need of treatment include subjects already suffering from a disorder or disease as well as subjects susceptible to a disorder or disease or subjects for whom prevention of a disorder or disease is intended. When referring to terms such as slow, moderate, reduce, ameliorate, and alleviate, their meanings also include elimination, disappearance, nonoccurrence, etc.

As used herein, the term “effective amount” refers to an amount of a therapeutic agent that is effective to prevent or alleviate symptoms of a disease or the progression of the disease when administered to a cell, tissue or subject alone or in combination with another therapeutic agent. “Effective amount” also refers to an amount of a compound that is sufficient to alleviate symptoms, e.g., to treat, cure, prevent, or alleviate the associated medical disorder, or to increase the rate at which such disorder is treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially or simultaneously.

As used herein, the term “cancer” refers to or describes a physiological condition in mammals that is typically characterized by unregulated cell growth. Included in this definition are benign and malignant cancers. As used herein, the term “tumor” or “neoplasm” refers to all neoplastic cell growth and proliferation, whether malignant or benign, and all pre-cancerous and cancerous cells and tissues. The terms “cancer” and “tumor” are not mutually exclusive when referred to herein.

### BRIEF DESCRIPTION OF THE DRAWINGS

The foregoing and other aspects of the present invention will be clearly explained by the following detailed description of the present invention and the accompanying drawings. The accompanying drawings herein are intended to illustrate certain preferred embodiments of the present invention. However, it should be understood that the present invention is not limited to the specific embodiments disclosed.

FIG. 1 shows the purity of human MSLN proteins determined by SDS-PAGE: M. Marker; 1. MSLN-R1-his: 4% non-reduced; 2.2 MSLN-R1-his: 8% non-reduced; 3. MSLN-R1-his: 16% non-reduced; 4. MSLN-R2-his: 8% non-reduced; 5. MSLN-R2-his: 16% non-reduced; 6. MSLN-R2-his: 50% non-reduced; 7. MSLN-R3-his: 16% non-reduced; 8. MSLN-R3-his: 50% non-reduced; 9. MSLN-FL-his: non-reduced; 10. MSLN-R3-rFc: non-reduced; 11. MSLN-FL-his: reduced; 12. MSLN-R3-rFc: reduced. The molecular weight of the Fc tag is about 50 KD, and lane 10 is the behavior of R3-rFc under a non-reduced condition, indicating the total molecular weight of protein +tag; and lane 12 is its behavior under a reduced condition. Because the reducing agent will break the disulfide bonds in the protein molecules and depolymerize into polypeptide chains, the reduced molecular weight is half of the non-reduced molecular weight.

FIG. 2 shows that: A is the binding activity of the human MSLN-R3-rFc protein and the anti-MSLN antibody determined by ELISA;
B is the binding activity of the human MSLN-FL-his protein and the anti-MSLN antibodies determined by ELISA;
C is the binding activity of the human MSLN-R1-his protein and the anti-MSLN antibodies determined by ELISA;
D is the binding activity of the human MSLN-R2-his protein and the anti-MSLN antibodies determined by ELISA; and
E is the binding activity of the human MSLN-R3-his protein and the anti-MSLN antibodies determined by ELISA.

FIG. 3 shows the binding activity of the control antibodies and the MSLN proteins determined by ELISA.

FIG. 4 shows that: A is the FACS result of the expression level of MSLN in Hela cells detected by using the control antibody Tab106;
B is the FACS result of the expression level of MSLN in Hela cells detected by using the control antibody Tab131; and
C is the FACS result of the expression level of MSLN in Hela cells detected by using the control antibody Tab142.

FIG. 5 shows that A is the FACS result of the expression level of MSLN in OVCAR3 cells detected by using the control antibody Tab106;
B is the FACS result of the expression level of MSLN in OVCAR3 cells detected by using the control antibody Tab131; and
C is the FACS result of the expression level of MSLN in OVCAR3 cells detected by using the control antibody Tab142.

FIG. 6 shows the FACS screening result of CHO-K1 cell strains transfected with the human MSLN proteins.

FIG. 7 shows the FACS result of the expression level of HEK293T cells transfected with a monkey MSLN protein using NB149 antiserum.

FIG. 8 shows the FACS screening result of HEK293T cell strains transfected with the human MSLN proteins.

FIG. 9 shows the FACS screening result of HEK293T cell strains transfected with the human MSLN-R3/chicken MSLN-R1-2 protein.

FIG. 10 shows the FACS screening result of HEK293T cell strains transfected with the human MSLN-R3 protein.

FIG. 11 shows that: A is the binding reaction of the control antibodies with human tumor cells OVCAR3 detected by FACS;
B is the binding reaction of the control antibodies with CHO-K1-human MSLN-2C8 cells detected by FACS; and
C is the binding reaction of the control antibodies with HEK293T-monkey MSLN cells detected by FACS.

FIG. 12 shows that: A is the antibody titer of serum of alpaca immunized with the human MSLN full-length protein using the human MSLN full-length protein;
B is the antibody titer of serum of alpaca immunized with the human MSLN full-length protein using the human MSLN-R3-his protein;
C is the antibody titer of serum of alpaca immunized with the human MSLN full-length protein using the human MSLN-R3-3 polypeptide; and
D is the antibody titer of serum of alpaca immunized with the human MSLN protein using Hela.

FIG. 13A shows the binding reaction of 20 nM VHH-hFc with the human MSLN proteins determined by ELISA; and

FIG. 13B shows the binding reaction of 0.2 nM VHH-hFc with the human MSLN proteins by ELISA.

FIG. 14 shows that A is the binding reaction of VHH-hFc with the human MSLN-FL-his protein determined by ELISA;
B is the binding reaction of VHH-hFc with the human MSLN-R1-his protein determined by ELISA;
C is the binding reaction of VHH-hFc with the human MSLN-R2-his protein determined by ELISA; and
D is the binding reaction of VHH-hFc with the human MSLN-R3-his protein determined by ELISA.

FIG. 15A shows the binding reaction of VHH-hFc with CHO-K1-human MSLN-2C8 cells detected by FACS; and

FIG. 15B shows the binding reaction of VHH-hFc with tumor cells Hela detected by FACS.

FIG. 16 shows that A is the binding reaction of VHH-hFc with tumor cells OVCAR3 detected by FACS,
B is the binding reaction of VHH-hFc with HEK293T-human MSLN-B8 cells detected by FACS; and
C is the binding reaction of VHH-hFc with HEK293T-human MSLN-R3/chicken MSLN-R1-2-A5 cells detected by FACS.

FIG. 17 shows the binding reaction of 20 nM VHH-hFc with HEK293T-human MSLN-B8, HEK293T-human MSLN-R3/chicken MSLN-R1-2-A5 cells detected by FACS.

FIG. 18 shows the specific binding reaction of VHH-hFc with tumor cells detected by FACS.

FIG. 19 shows the binding reaction of VHH-hFc with HEK293T-monkey MSLN cells detected by FACS

FIG. 20 shows the affinity of VHH-hFc for the human MSLN-FL-his protein determined by SPR.

FIG. 21 shows the inhibition rate of VHH-hFc by the competitive ELISA method.

FIG. 22A shows the competitive activity between VHH-hFc and Biotin-Tab142; and

FIG. 22B shows the competitive activity between VHH-hFc and Biotin-Tab131.

FIG. 23 shows the epitope classification of VHH-hFc.

### DETAILED DESCRIPTION

The present invention will be described in detail below with reference to examples and the accompanying drawings. The accompanying drawings herein are intended to illustrate some preferred embodiments of the present invention. However, it should be understood that the present invention is not limited to the specific embodiments disclosed or they are not regarded as a limitation to the scope of the present invention. Experimental procedures without specified conditions in the examples are conducted according to conventional conditions or conditions recommended by the manufacturers. Reagents or instruments without specified manufacturers used herein are conventional products that are commercially available.

### Example 1: Preparation of Control Antibodies, Identification of Endogenous Cells and Preparation of Overexpression Cell Strains

(A) Preparation of Control Antibodies

The YP218, YP3 and YP223 sequences were from the patent US2015252118A1, the m912 sequence was from the patent WO2009120769A1, and the Amatuximab (recognizing the epitope of human MSLN R1) sequence was from the patent US20140127237A1. VH and VL sequences of clone YP218 recognizing the epitope of human MSLN R3 and clone YP3 recognizing the conformational epitope of human MSLN were recombined into human IgG1 CH and CL expression vectors; VH and VL sequences of clone YP223 recognizing the epitope of human MSLN R2 were recombined into rabbit IgG1 CH and CL expression vectors; and VH and VL of clones m912 and YP218 recognizing the epitope of human MSLN R3 were linked by 3 GGGGS linkers and then recombined into a human IgG1 Fc expression vector to give a recombinant plasmid. Both the construction of plasmid and the expression and purification of antibodies were carried out by Biointron Biological Inc.

Amatuximab, a YP218 human IgG1 antibody, a YP223 rabbit IgG1 antibody, a YP3 human IgG1 antibody, a YP218 scFv-human IgG1 Fc (hFc) antibody and an m912 scFv-human IgG1 Fc (hFc) antibody were named Tab142 (Amatuximab), Tab106 (YP218, hIgG1), Tab020 (YP223, rabbitIgG1), Tab107 (YP3, hIgG1), Tab108 (YP218, scFv-hIgG1 Fc) and Tab131 (m912, scFv-hIgG1 Fc), respectively.

TABLE 1
Sequence Information of Control Antibodies

| | Sequence | |
|---|---|---|
| Sequence | No. | Amino Acid Sequence |
| YP223 VH | SEQ ID | QEQLEESGGDLVQPEGSLTL |
| | NO: 1 | TCKASGLDFSSSYWICWVRQ |
| | | APGKGLEWIGCRHTFTANTW |
| | | SASWVNGRFTISRSTSLGTV |
| | | DLKMTSLTAADTATYFCARD |
| | | ESNNDGWDFKLWGPGTLVTV |
| | | SS |
| YP223 VL | SEQ ID | AYDMTQTPASVSAAVGGTVT |
| | NO: 2 | IKCQASQSISNYLAWYQQKP |
| | | GQPPKLLIYQASTLAPGVSS |
| | | RFKGSGSGTEFTLTISGVEC |
| | | ADAATYYCQQGYTSSNVENV |
| | | FGGGTGVVV |
| YP218 VH | SEQ ID | QQQLEESGGGLVKPEGSLTL |
| | NO: 3 | TCKASGFDLGFYFYACWVRQ |
| | | APGKGLEWIACIYTAGSGST |
| | | YYASWAKGRFTISKASSTTV |
| | | TLQMTSLAAADTATYFCARS |
| | | TANTRSTYYLNLWGPGTLV |
| | | TVSS |
| YP218 VL | SEQ ID | DVVMTQTPASVSEPVGGTVT |
| | NO: 4 | IKCQASQRISSYLSWYQQKP |
| | | GQRPKLLIFGASTLASGVPS |
| | | RFKGSGSGTEYTLTISDLEC |
| | | ADAATYYCQSYAYFDSNNWH |
| | | AFGGGTEVVV |
| YP3 VH | SEQ ID | QEQLVESGGGLVQPGASLTL |
| | NO: 5 | TCTASGIDFSRYYMCWVRQA |
| | | PGKGLEGIACIYIGGSGSTY |
| | | YASWAKGRFTISKASSTTVT |
| | | LQMTSLTAADTATYFCARGT |
| | | NLNYIFRLWGPGTLVTVSS |
| YP3 VL | SEQ ID | DVVMTQTPSPVSAAVGGTVT |
| | NO: 6 | IKCQASQSINNGLAWYQQKP |
| | | GQPPRLLIYSASNLESGVPS |
| | | RFKGSGSGTEFTLTISDLEC |
| | | DDAATYYCQCIWDGNSYVNA |
| | | FGGGTEVVV |
| m912 scFv | SEQ ID | QVQLQESGPGLVKPSETLSL |
| | NO: 7 | TCTVSGGSVSSGSYYWSWIR |
| | | QPPGKGLEWIGYIYYSGSTN |
| | | YNPSLKSRVTISVDTSKNQF |
| | | SLKLSSVTAADTAVYYCARE |
| | | GKNGAFDIWGQGTMVTVSSS |
| | | GGGGSGGGGSGGGGSRHQMT |
| | | QSPSSLSASVGDRVTITCRA |
| | | SQSISSYLNWYQQKPGKAPK |
| | | LLIYAASSLQSGVPSRFSGS |
| | | GSGTDFTLTISSLQPEDFAT |
| | | YYCQQSYSTPLTFGGGTKVE |
| | | IKGQAG |
| YP218 scFv | SEQ ID | QQQLEESGGGLVKPEGSLTL |
| | NO: 8 | TCKASGFDLGFYFYACWVRQ |
| | | APGKGLEWIACIYTAGSGST |
| | | YYASWAKGRFTISKASSTTV |
| | | TLQMTSLAAADTATYFCARS |
| | | TANTRSTYYLNLWGPGTLVT |
| | | VSSGGGGSGGGGSGGGGSDV |
| | | VMTQTPASVSEPVGGTVTIK |
| | | CQASQRISSYLSWYQQKPGQ |
| | | RPKLLIFGASTLASGVPSRF |
| | | KGSGSGTEYTLTISDLECAD |
| | | AATYYCQSYAYFDSNNWHA |
| | | FGGGTEVVV |
| Amatuximab VH- | SEQ ID | QVQLQQSGPELEKPGASVKI |
| CH | NO: 9 | SCKASGYSFTGYTMNWVKQS |
| | | HGKSLEWIGLITPYNGASSY |
| | | NQKFRGKATLTVDKSSSTAY |
| | | MDLLSLTSEDSAVYFCARGG |
| | | YDGRGFDYWGSGTPVTVSSA |
| | | STKGPSVFPLAPSSKSTSGG |
| | | TAALGCLVKDYFPEPVTVSW |
| | | NSGALTSGVHTFPAVLQSSG |
| | | LYSLSSVVTVPSSSLGTQTY |
| | | ICNVNHKPSNTKVDKKVEPK |
| | | SCDKTHTCPPCPAPELLGGP |
| | | SVFLFPPKPKDTLMISRTPE |
| | | VTCVVVDVSHEDPEVKFNWY |
| | | VDGVEVHNAKTKPREEQYNS |
| | | TYRVVSVLTVLHQDWLNGKE |
| | | YKCKVSNKALPAPIEKTISK |
| | | AKGQPREPQVYTLPPSRDEL |
| | | TKNQVSLTCLVKGFYPSDIA |
| | | VEWESNGQPENNYKTTPPVL |
| | | DSDGSFFLYSKLTVDKSRWQ |
| | | QGNVFSCSVMHEALHNHYTQ |
| | | KSLSLSPGK |
| Amatuximab VL- | SEQ ID | DIELTQSPAIMSASPGEKVT |
| CL | NO: 10 | MTCSASSSVSYMHWYQQKSG |
| | | TSPKRWIYDTSKLASGVPGR |
| | | FSGSGSGNSYSLTISSVEAE |
| | | DDATYYCQQWSKHPLTFGSG |
| | | TKVEIKRTVAAPSVFIFPPS |
| | | DEQLKSGTASVVCLLNNFYP |
| | | REAKVQWKVDNALQSGNSQE |
| | | SVTEQDSKDSTYSLSSTLTL |
| | | SKADYEKHKVYACEVTHQGL |
| | | SSPVTKSFNRGEC |
| hFc | SEQ ID | EPKSADKTHTCPPCPAPELL |
| | NO: 11 | GGPSVFLFPPKPKDTLMISR |
| | | TPEVTCVVVDVSHEDPEVKF |
| | | NWYVDGVEVHNAKTKPREEQ |
| | | YNSTYRVVSVLTVLHQDWLN |
| | | GKEYKCKVSNKALPAPIEKT |
| | | ISKAKGQPREPQVYTLPPSR |
| | | EEMTKNQVSLTCLVKGFYP |
| | | SDIAVEWESNGQPENNYKT |
| | | TPPVLDSDGSFFLYSKLTVD |
| | | KSRWQQGNVFSCSVMHEALH |
| | | NHYTQKSLSLSPGK |
| CH-hIgG1 | SEQ ID | ASTKGPSVFPLAPSSKSTSG |
| (human IgG1 | NO: 12 | GTAALGCLVKDYFPEPVTVS |
| heavy chain | | WNSGALTSGVHTFPAVLQSS |
| constant | | GLYSLSSVVTVPSSSLGTQT |
| region) | | YICNVNHKPSNTKVDKKVEP |
| | | KSCDKTHTCPPCPAPELLGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| CL-hIgG1 | SEQ ID | RTVAAPSVFIFPPSDEQLKS |
| (human IgG1 | NO: 13 | GTASVVCLLNNFYPREAKVQ |
| light chain | | WKVDNALQSGNSQESVTEQD |
| constant | | SKDSTYSLSSTLTLSKADYE |
| region) | | KHKVYACEVTHQGLSSPVTK |
| | | SFNRGEC |
| CH-rabbit | SEQ ID | GQPKAPSVFPLAPCCGDTPS |
| IgG1 | NO: 14 | STVTLGCLVKGYLPEPVTVT |
| (rabbit IgG1 | | WNSGTLTNGVRTFPSVRQSS |
| heavy chain | | GLYSLSSVVSVTSSSQPVTC |
| constant | | NVAHPATNTKVDKTVAPSTC |
| region) | | SKPTCPPPELLGGPSVFIFP |
| | | PKPKDTLMISRTPEVTCVVV |
| | | DVSQDDPEVQFTWYINNEQV |
| | | RTARPPLREQQFNSTIRVVS |
| | | TLPIAHQDWLRGKEFKCKVH |
| | | NKALPAPIEKTISKARGQPL |
| | | EPKVYTMGPPREELSSRSVS |
| | | LTCMINGFYPSDISVEWEKN |
| | | GKAEDNYKTTPAVLDSDGSY |
| | | FLYSKLSVPTSEWQRGDVFT |
| | | CSVMHEALHNHYTQKSISRS |
| | | PGK |
| CL-rabbit | SEQ ID | GDPVAPTVLIFPPAADQVAT |
| IgG1 | NO: 15 | GTVTIVCVANKYFPDVTVTW |
| (rabbit | | EVDGTTQTTGIENSKTPQNS |
| IgG1 light | | ADCTYNLSSTLTLTSTQYNS |
| chain | | HKEYTCKVTQGTTSVVQSFN |
| constant | | RGDC |
| region) | | |

(B) Preparation of Human MSLN-R3-rFc, MSLN-FL-his, MSLN-R1-his, MSLN-R2-his and MSLN-R3-his:

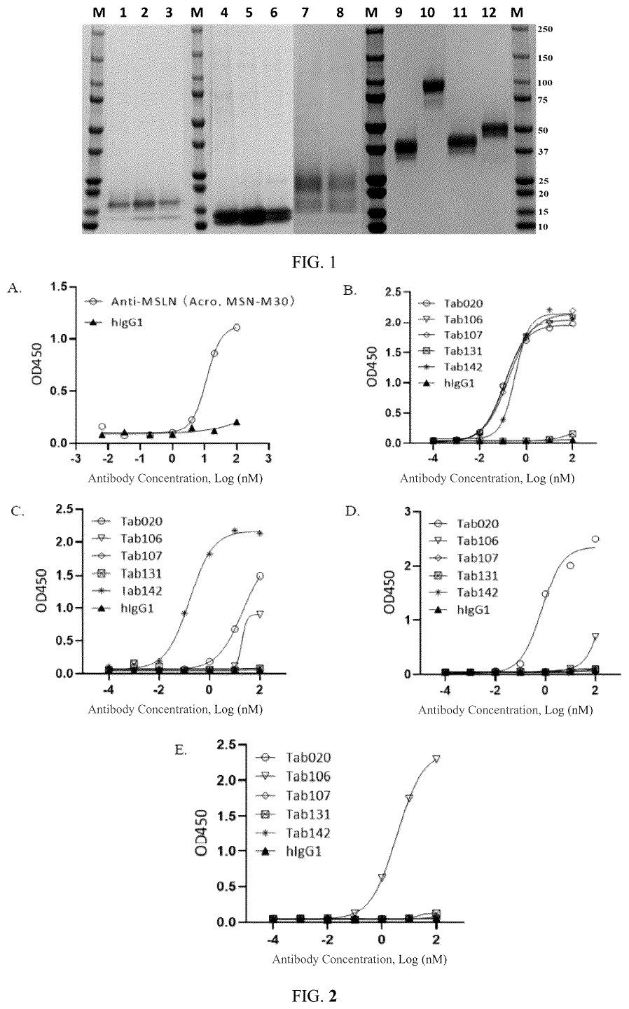

The MSLN protein has 3 IgG-like domains extracellularly, with Region1 (R1) being located at the most distal end of the membrane, Region3 (R3) at the most proximal end of the membrane, the antigen-binding epitope of Amatuximab being located at R1, and YP218 being located at R3. Nucleotide sequences which contain the nucleotide sequences encoding human MSLN protein extracellular domain amino acid sequences Glu296-Gly580 (MSLN-FL), Glu296-Thr390 (MSLN-R1), Ser391-Asn486 (MSLN-R2) and Met487-Ser598 (MSLN-R3) were cloned into a pTT5 vector (manufactured by General Biol (Anhui) Co., Ltd), respectively, and plasmids were prepared according to an established standard molecular biology method, wherein the rFc represents an Fc tag from a rabbit antibody, and the his is a histidine tag. Detailed information can be obtained by referring to Sambrook, J., Fritsch, E. F., and Maniatis, T. (1989). Molecular Cloning: A Laboratory Manual, Second whereinEdition (Plainview, New York: Cold Spring Harbor Laboratory Press). HEK293E cells (purchased from Suzhou Yiyan Biotech Co., Ltd.) were transiently transfected (PEI, Polysciences, Cat. No. 24765-1) and expanded at 37° C. using FreeStyle™ 293 (Thermofisher scientific, Cat. No. 12338018). After 6 days, the cell culture medium was collected and centrifuged to remove cell components to give a culture supernatant containing the extracellular domain of the human MSLN protein. The culture supernatant was loaded onto a nickel ion affinity chromatography column HisTrap™ Excel (GE Healthcare, Cat. No. GE17-3712-06), and meanwhile, the changes in UV absorption value (A280 nm) were monitored using an ultraviolet (UV) detector. After loading, the nickel ion affinity chromatography column was washed with 20 mM PB, 0.5 M NaCl (pH 7.4) until the UV absorption value returned to baseline, gradient elution (2%, 4%, 8%, 16%, 50%, 100%) was then performed with buffer A: 20 mM PB, 0.5 M NaCl (pH 7.4) and buffer B: 20 mM PB, 0.5 M NaCl, 500 mM imidazole, and the human MSLN protein with the His tag eluted from the nickel ion affinity chromatography column was collected. The culture supernatant was loaded onto a protein A chromatography column (the protein A filler AT Protein A Diamond and the chromatography column BXK16/26 were both purchased from Bestchrom), the protein A chromatography column was washed with PBS phosphate buffer (pH 7.4) and 20 mM PB, 1 M NaCl (pH 7.2) in sequence, and was finally eluted with citric acid buffer (pH 3.4), and the human MSLN protein with the rabbit Fc (rFc) tag eluted from the protein A chromatography column was collected. Dialysis was performed with PBS phosphate buffer (pH 7.4) at 4° C. overnight in a refrigerator. The dialyzed protein was subjected to 0.22 μM sterile filtration, subpackaged, and stored at −80° C., giving a purified human MSLN extracellular domain protein. The target bands of the sample as assayed by SDS-PAGE reduced gel and non-reduced gel are shown in FIG. 1.

Human MSLN protein (NCBI: AAH09272.1,
SEQ ID NO: 16):
MALPTARPLLGSCGTPALGSLLFLLFSLGWVQPSRTLAGE
TGQEAAPLDGVLANPPNISSLSPRQLLGFPCAEVSGLSTE
RVRELAVALAQKNVKLSTEQLRCLAHRLSEPPEDLDALPL
DLLLFLNPDAFSGPQACTRFFSRITKANVDLLPRGAPERQ
RLLPAALACWGVRGSLLSEADVRALGGLACDLPGRFVAES
AEVLLPRLVSCPGPLDQDQQEAARAALQGGGPPYGPPSTW
SVSTMDALRGLLPVLGQPIIRSIPQGIVAAWRQRSSRDPS
WRQPERTILRPRFRREVEKTACPSGKKAPEIDESLIFYKK
WELEACVDAALLATQMDRVNAIPFTYEQLDVLKHKLDELY
PQGYPESVIQHLGYLFLKMSPEDIRKWNVTSLETLKALLE
VNKGHEMSPQVATLIDRFVKGRGQLDKDTLDTLTAFYPGY
LCSLSPEELSSVPPSSIWAVRPQDLDTCDPRQLDVLYPKA
RLAFQNMNGSEYFVKIQSFLGGAPTEDLKALSQQNVSMDL
ATFMKLRTDAVLPLTVAEVQKLLGPHVEGLKAEERHRPVR
DWILRQRQDDLDTLGLGLQGGIPNGYLVLDLSMQEALSGT
PCLLGPGPVLTVLALLLASTLA

The prepared human MSLN proteins described above were detected by ELISA using positive control antibodies recognizing different epitopes, and the detection results are shown in FIG. 2 and Tables 2-6. The human MSLN-R3-rFc, MSLN-FL-his, MSLN-R1-his, MSLN-R2-his and MSLN-R3-his proteins have binding activity to the anti-human MSLN antibody (purchased from Acro, Cat. No. MSN-M30) or the control antibodies, and are consistent with the binding epitopes of Tab142 (Amatuximab), Tab106 (YP218), Tab020 (YP223) and Tab107 (YP3) reported in the product specifications or literature, which indicates that the aforementioned proteins with binding activity have been prepared.

TABLE 2
Binding Reaction of Human MSLN-R3-rFc
Protein with Antibodies Determined by ELISA
OD450 nm

| Concen- | Antibody | |
|---|---|---|
| tration | anti-hMSLN | |
| (nM) | (purchased from Acro) | hIgG1 |
| 100 | 1.11 | 0.21 |
| 20 | 0.86 | 0.12 |
| 4 | 0.23 | 0.15 |
| 1 | 0.11 | 0.09 |
| 0.2 | 0.08 | 0.08 |
| 0.032 | 0.07 | 0.11 |
| 0.0064 | 0.16 | 0.08 |
| 0 | 0.07 | 0.08 |

TABLE 3
Binding Reaction of Human MSLN-FL-his
Protein with Antibodies Determined by ELISA
OD450 nm

| Concentra- | Antibody | | | | | |
|---|---|---|---|---|---|---|
| tion (nM) | Tab020 | Tab106 | Tab107 | Tab131 | Tab142 | hIgG1 |
| 100 | 1.98 | 2.08 | 2.20 | 0.16 | 2.08 | 0.06 |
| 10 | 1.90 | 1.99 | 2.00 | 0.07 | 2.21 | 0.05 |
| 1 | 1.70 | 1.76 | 1.74 | 0.05 | 1.79 | 0.05 |
| 0.1 | 0.95 | 0.92 | 0.85 | 0.04 | 0.39 | 0.05 |
| 0.01 | 0.18 | 0.16 | 0.15 | 0.04 | 0.15 | 0.05 |
| 0.001 | 0.06 | 0.05 | 0.06 | 0.05 | 0.05 | 0.05 |
| 0.0001 | 0.05 | 0.04 | 0.05 | 0.05 | 0.05 | 0.04 |
| 0 | 0.05 | 0.05 | 0.05 | 0.05 | 0.05 | 0.05 |

TABLE 4
Binding Reaction of Human MSLN-R1-his
Protein with Antibodies Determined by ELISA
OD450 nm

| Concentra- | Antibody | | | | | |
|---|---|---|---|---|---|---|
| tion (nM) | Tab020 | Tab106 | Tab107 | Tab131 | Tab142 | hIgG1 |
| 100 | 1.49 | 0.90 | 0.08 | 0.09 | 2.14 | 0.06 |
| 10 | 0.68 | 0.12 | 0.06 | 0.05 | 2.18 | 0.05 |
| 1 | 0.19 | 0.06 | 0.05 | 0.06 | 1.82 | 0.05 |
| 0.1 | 0.07 | 0.05 | 0.05 | 0.05 | 0.92 | 0.05 |
| 0.01 | 0.05 | 0.04 | 0.06 | 0.11 | 0.19 | 0.05 |
| 0.001 | 0.10 | 0.17 | 0.05 | 0.14 | 0.07 | 0.05 |
| 0.0001 | 0.05 | 0.04 | 0.04 | 0.05 | 0.11 | 0.05 |
| 0 | 0.06 | 0.05 | 0.05 | 0.05 | 0.05 | 0.05 |

TABLE 5
Binding Reaction of Human MSLN-R2-his
Protein with Antibodies Determined by ELISA
OD450 nm

| Concentra- | Antibody | | | | | |
|---|---|---|---|---|---|---|
| tion (nM) | Tab020 | Tab106 | Tab107 | Tab131 | Tab142 | hIgG1 |
| 100 | 2.50 | 0.70 | 0.09 | 0.10 | 0.11 | 0.06 |
| 10 | 2.01 | 0.11 | 0.05 | 0.05 | 0.09 | 0.05 |
| 1 | 1.49 | 0.05 | 0.05 | 0.04 | 0.07 | 0.05 |
| 0.1 | 0.20 | 0.05 | 0.04 | 0.05 | 0.05 | 0.05 |
| 0.01 | 0.07 | 0.04 | 0.04 | 0.04 | 0.04 | 0.05 |
| 0.001 | 0.05 | 0.04 | 0.05 | 0.04 | 0.05 | 0.05 |
| 0.0001 | 0.05 | 0.04 | 0.04 | 0.04 | 0.05 | 0.05 |
| 0 | 0.05 | 0.05 | 0.05 | 0.05 | 0.05 | 0.04 |

TABLE 6
Binding Reaction of Human MSLN-R3-his
Protein with Antibodies Determined by ELISA
OD450 nm

| Concentra- | Antibody | | | | | |
|---|---|---|---|---|---|---|
| tion (nM) | Tab020 | Tab106 | Tab107 | Tab131 | Tab142 | hIgG1 |
| 100 | 0.05 | 2.29 | 0.09 | 0.12 | 0.05 | 0.05 |
| 10 | 0.05 | 1.75 | 0.05 | 0.05 | 0.05 | 0.05 |
| 1 | 0.05 | 0.62 | 0.05 | 0.04 | 0.04 | 0.05 |
| 0.1 | 0.05 | 0.12 | 0.05 | 0.04 | 0.05 | 0.05 |
| 0.01 | 0.05 | 0.06 | 0.04 | 0.03 | 0.04 | 0.05 |
| 0.001 | 0.05 | 0.05 | 0.05 | 0.05 | 0.05 | 0.04 |
| 0.0001 | 0.05 | 0.05 | 0.05 | 0.05 | 0.05 | 0.04 |
| 0 | 0.05 | 0.05 | 0.05 | 0.05 | 0.05 | 0.05 |

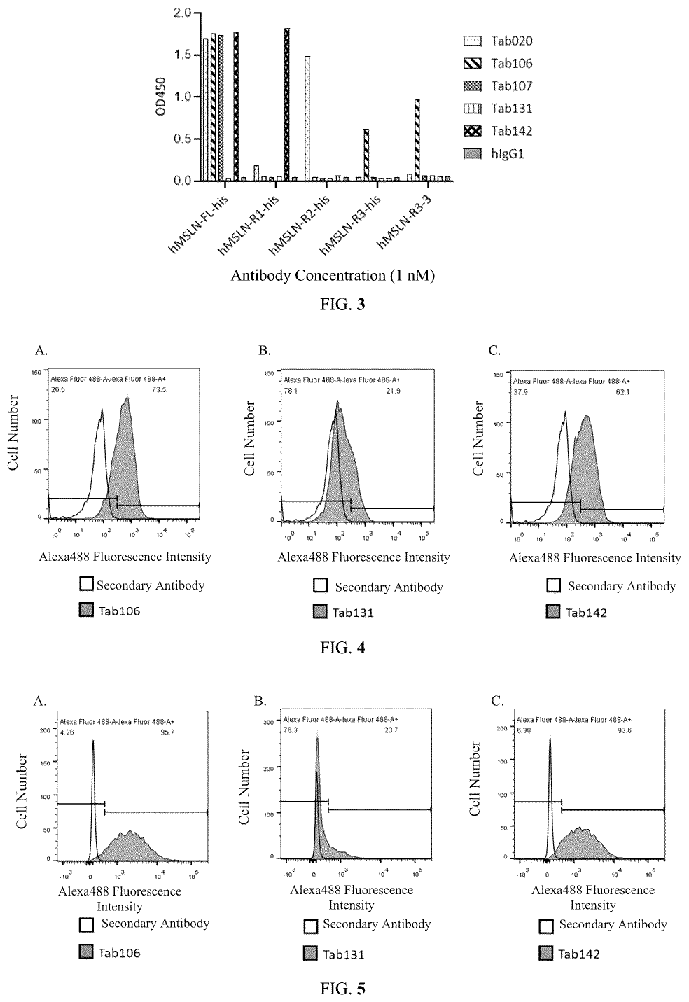

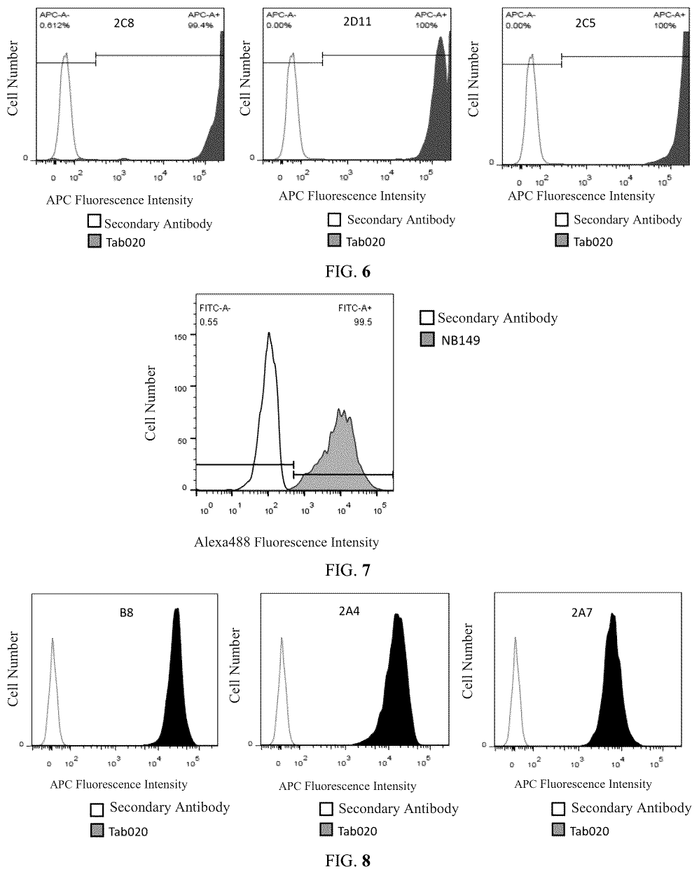

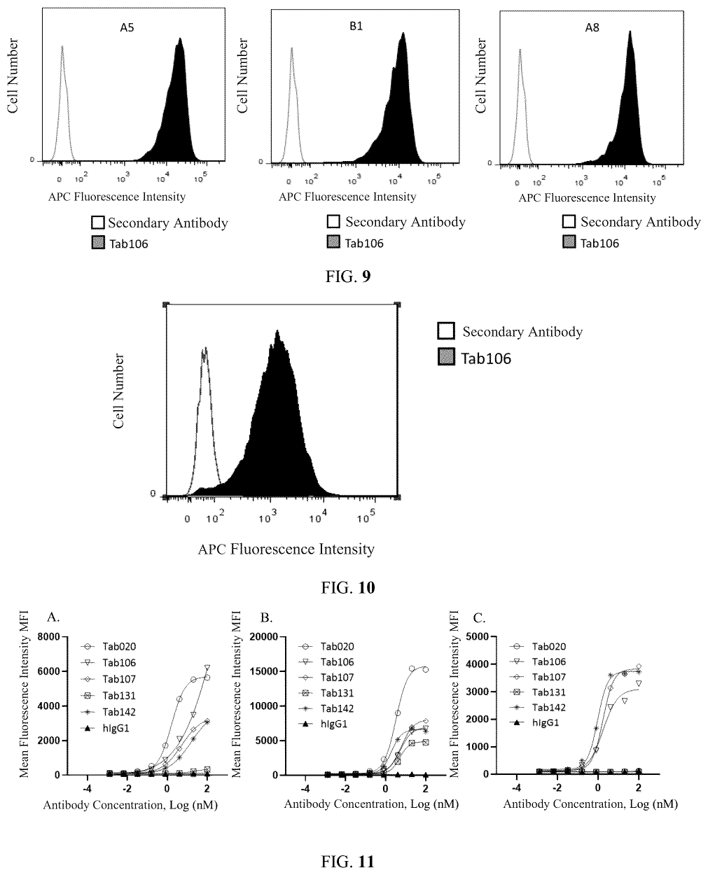

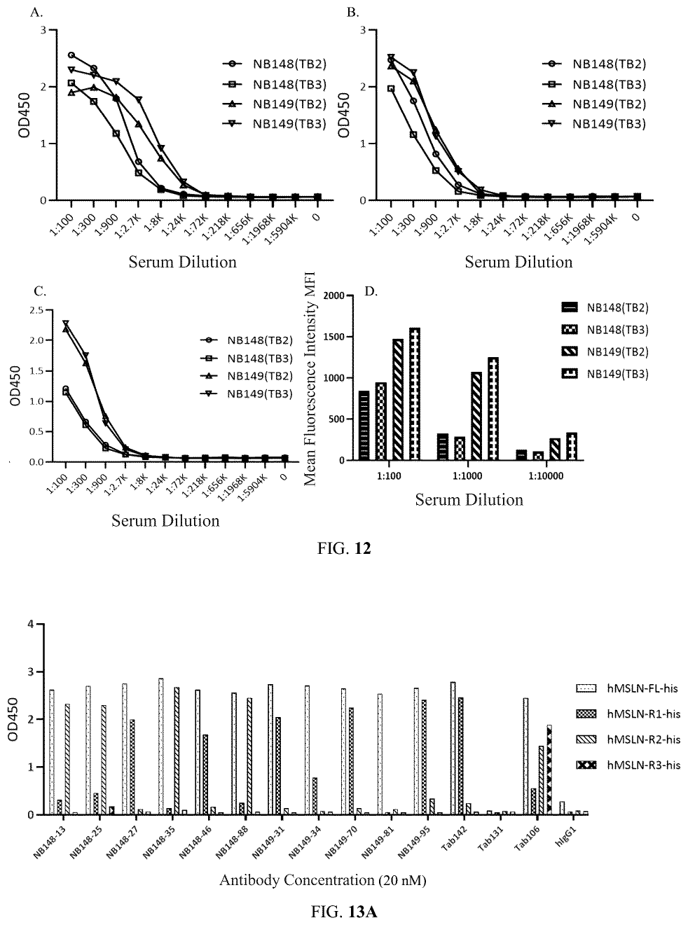

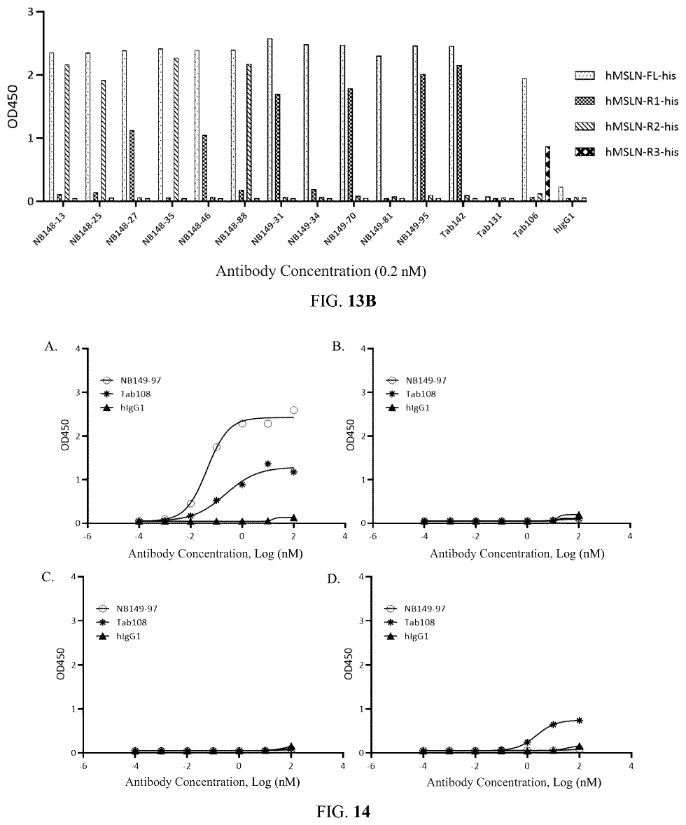

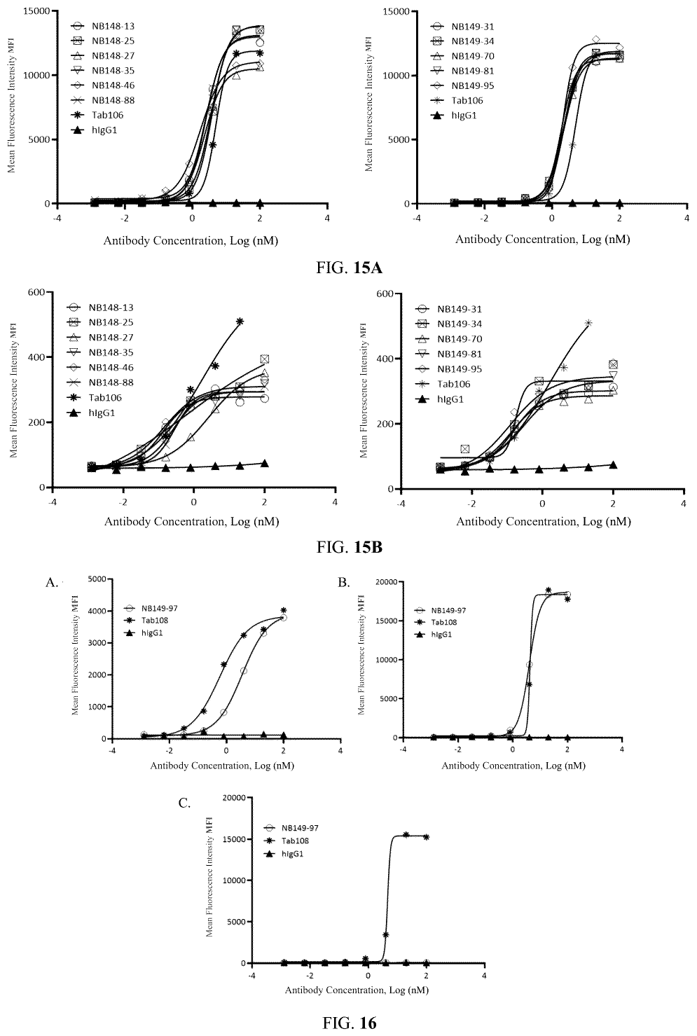

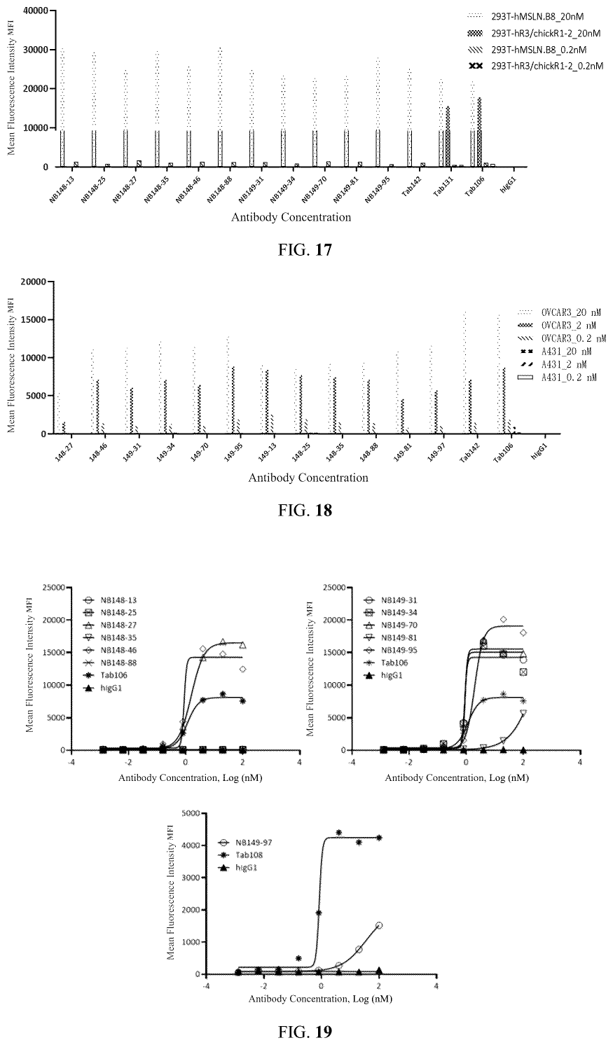

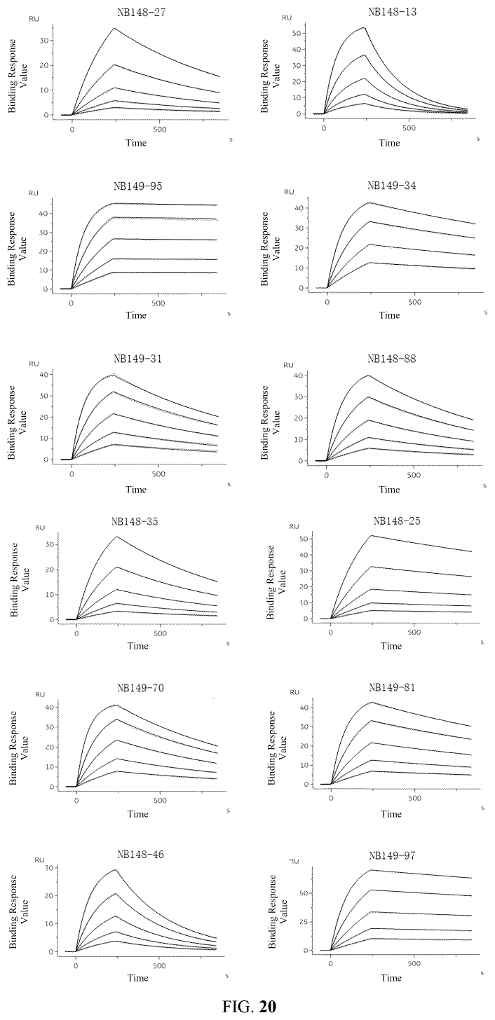

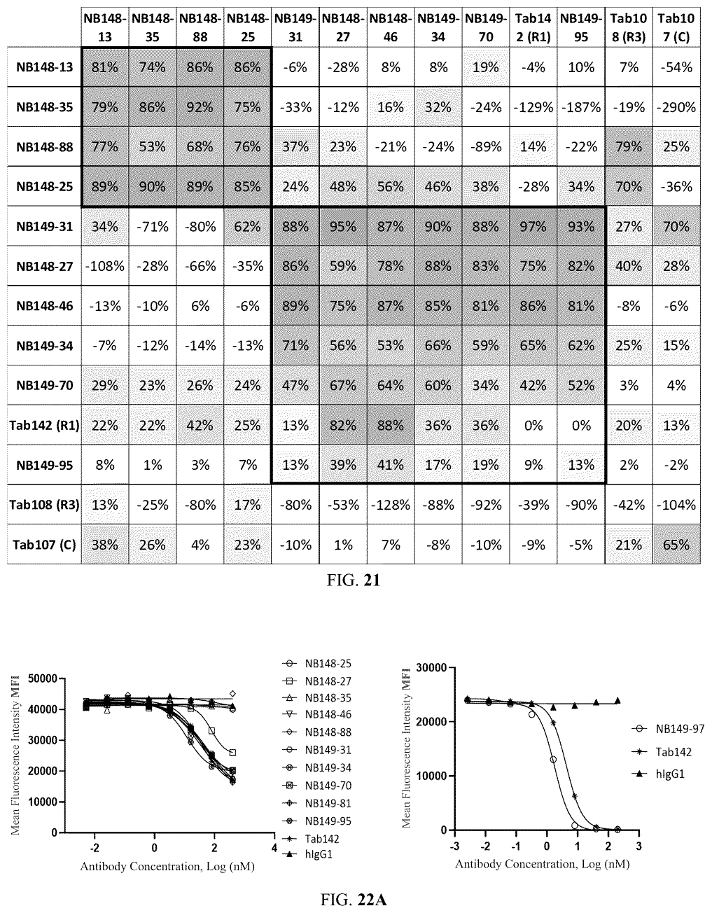

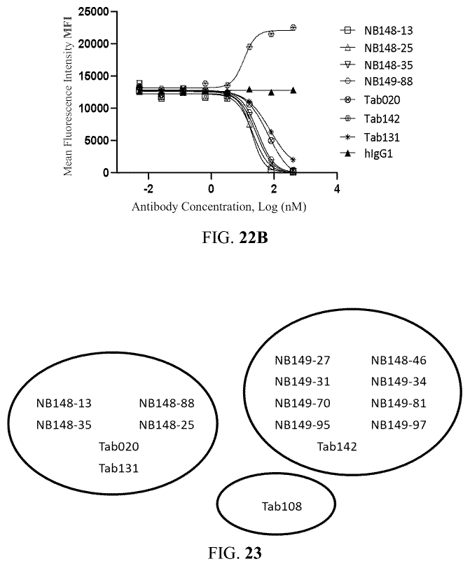

The binding activities of the control antibodies with the human MSLN-FL-His protein, MSLN-R1-His protein, MSLN-R2-His protein, MSLN-R3-His protein and MSLN-R3-3 polypeptide (purchased from GL Biochem, Cat. No. 406676) are shown in Table 7 and FIG. 3, and the results show that the Tab020 (YP223), Tab142 (Amatuximab), Tab106 (YP218) and Tab107 (YP3) antibodies have good binding activities to the human MSLN-FL-His protein and that Tab131 (m912 scFv-hFc) have almost no binding activity to the human MSLN-FL-His and MSLN-R3-His proteins under the same experimental conditions.

TABLE 7
Binding Reaction of Control Antibodies with
Human MSLN-FL-his Protein Determined by ELISA
OD450 nm

| | Antibody (1 nM) | | | | | |
|---|---|---|---|---|---|---|
| Antigen | Tab020 | Tab106 | Tab107 | Tab131 | Tab142 | hIgG1 |
| hMSLN-FL-his | 1.70 | 1.76 | 1.74 | 0.05 | 1.79 | 0.05 |
| hMSLN-R1-his | 0.19 | 0.06 | 0.05 | 0.06 | 1.82 | 0.05 |
| hMSLN-R2-his | 1.49 | 0.05 | 0.05 | 0.04 | 0.07 | 0.05 |
| hMSLN-R3-his | 0.05 | 0.62 | 0.05 | 0.04 | 0.04 | 0.05 |
| hMSLN-R3-3 | 0.09 | 0.98 | 0.07 | 0.07 | 0.06 | 0.06 |

(C) Identification of Cell Strain Endogenously Expressing Human MSLN Protein

Cells endogenously expressing a human MSLN protein were expanded to the logarithmic growth phase in a T-75 cell culture flask, medium supernatant was discarded by centrifugation, and the cell pellet was washed twice with PBS. 20 nM Tab106, Tab131 and Tab142 antibodies were used as primary antibodies, and FITC-labeled secondary antibodies (purchased from Invitrogen, Cat. No. A18830) were detected and analyzed by FACS (FACS Canto™, purchased from BD). The results are shown in Table 8, FIG. 4 and FIG. 5, indicating that the cells endogenously expressing the human MSLN protein have binding activity to all of Tab106, Tab131 and Tab142.

TABLE 8
FACS Result of Expression Level of MSLN in Tumor Cells

| | | Mean Fluorescence Intensity | | | |
|---|---|---|---|---|---|
| | Endogenously | Secondary | | | |
| | Expressing | Antibody | | | |
| No. | Cell Line | Control | Tab106 | Tab131 | Tab142 |
| 1 | Hela | 71 | 647 | 219 | 533 |
| 2 | OVCAR3 | 75 | 3484 | 211 | 2096 |

(D) Preparation of CHO-K1 Recombinant Cell Strains Expressing Human MSLN Full-length Protein

A nucleotide sequence encoding a full-length amino acid sequence of human MSLN (NCBI: AAH09272.1, SEQ ID NO: 16) was cloned into a pcDNA3.1 vector, and a plasmid was prepared (performed by General Biol (Anhui) Co., Ltd). After the plasmid transfection (Lipofectamine® 3000 Transfection Kit, purchased from Invitrogen, Cat. No. L3000-015) of a CHO-K1 cell line (purchased from Shanghai Institutes for Biological Sciences), selective culture was performed in DMEM/F12 medium containing 10 μg/mL of puromycin and 10% (w/w) of fetal bovine serum for 2 weeks, and positive monoclonal cells were sorted into 96 well plates on a flow cytometer FACSACriaII (purchased from BD Biosciences) using the rabbit anti-human MSLN antibody (Tab020) and a goat anti-rabbit IgG Fab antibody (cell signaling, Cat. No. 4414S) and cultured under the conditions of 37° C. and 5% (v/v) CO₂. After about 2 weeks, some of the monoclonal wells were selected for amplification. The amplified clones were screened by flow cytometry. Monoclonal cell lines with better growth and higher fluorescence intensity were selected for further expansion and cryopreserved in liquid nitrogen.

The specific selection results are shown in Table 9 and FIG. 6, and only the secondary antibody was incubated as a control. Table 9 indicates that a series of CHO-K1 monoclonal cell lines positively expressing human MSLN have been prepared. In FIG. 6, the abscissa represents the fluorescence intensity of the cells, and the ordinate represents the number of the cells. The results indicate that 2C8, 2D11 and 2C5 are recombinant CHO-K1 cell strains which highly express the human MSLN proteins.

TABLE 9
FACS Result of CHO-K1 Recombinant Cell Lines
Expressing Human MSLN Full-length Protein

| | | Mean Fluorescence Intensity of Cells | |
|---|---|---|---|
| | Clone No. of Stably | Secondary | Tab020 |
| No. | Transfected Cell Line | Antibody Control | Antibody |
| 1 | CHO-K1-hMSLN-2C8 | 37 | 233609 |
| 2 | CHO-K1-hMSLN-2D11 | 37 | 217642 |
| 3 | CHO-K1-hMSLN-2C5 | 37 | 205726 |

(E) Preparation of Recombinant HEK293T Cell Strains Expressing Monkey MSLN Protein

A nucleotide sequence encoding a full-length amino acid sequence of monkey MSLN (NCBI: XP_028696439.1, SEQ ID NO: 17) was cloned into a pcDNA3.1 vector, and a plasmid was prepared. After the plasmid transfection (Lipofectamine® 3000 Transfection Kit, purchased from Invitrogen, Cat. No. L3000-015) of an HEK293T cell line (purchased from ATCC), selective culture was performed in DMEM/F12 medium containing 10 μg/ml of puromycin and 10% (w/w) of fetal bovine serum for 2 weeks, subcloning was performed in 96-well culture plates by the limiting dilution method, and culturing was performed under the conditions of 37° C. and 5% (v/v) CO₂. After about 2 weeks, some of the polyclonal wells were selected for amplification in 6-well plates. The amplified clones were examined and analyzed by an FACS flow cytometer using NB149 antiserum (for antiserum preparation, see Example 2), and the cell strains with better growth and higher fluorescence intensity were selected for continuous expansion and cryopreserved in liquid nitrogen. The results of expression levels are shown in Table 10 and FIG. 7, showing that HEK293T-monkey-MSLN screened with puromycin under pressure has a relatively single positive peak, and can be used for the detection of cross activity of antibodies to monkey MSLN proteins by FACS.

Full-length amino acid sequence of
monkey MSLN (NCBI: XP-028696439.1,
SEQ ID NO: 17):
MALPMARPLSGSCGTPAVGSLLFLLFSLGWVQPSRVLAGE
TRQALCPQEAAPLDGILTNAPDIASLSPRQLLGFTCVEVS
GLSTELVQELAVALGQKNVKLSAEQLRCLAHRLSEPPEDL
DALPLDLLLFLNPDAFSGPQACTHFFSRVAKANVDLLPRG
APERQRLLPAALTCWGVRGSLLSEADVRALGGLACDLPGC
FVAESAEVVLPRLVRCLGPLDQDQQEAARAALQRGGPPYG
PPSTWSISTLDDLQSLLPVLGQPVIHSIPKGILAAWRQRS
SRDPSWQQPEQTVLRPRFRRDVERTTCPPEKEVHEIDESL
IFYKKRELEACVDAALLAAQMDRVDAIPFTYEQLDVLKHK
LDELYPQGYPESVIRHLGHLFLKMSPEDIRKWNVTSLETL
KALLKVSKGHEMSAQVATLIDRVVVGRGQLDKDTVDTLTA
FCPGCLCSLSPERLSSVPPSVIGAVRPQDLDTCGPRQLDV
LYPKARLAFQNMSGSEYFVKIRPFLGGAPTEDVKALSQQN
VSMDLATFMKLRREAVLPLTVAEVQKLLGPHVEGLKVEEQ
HSPVRDWILKQRQDDLDTLGLGLQGGIPNGYLILDLSVRE
ALSGTPCLLGPGPVLTVLALLLASTLA

TABLE 10
FACS Result of HEK293T Recombinant Cell Lines
Expressing Monkey MSLN Full-length Protein

| | Clone No. of | Mean Fluorescence Intensity of Cells | |
|---|---|---|---|
| | Stably Transfected | Secondary | NB149 |
| No. | Cell Line | Antibody Control | Antiserum |
| 1 | HEK293T- | 105 | 12423 |
| | monkey MSLN | | |

(F) Preparation of Recombinant HEK293T Cell Strains Expressing Human MSLN Protein

A nucleotide sequence encoding a full-length amino acid sequence of human MSLN (NCBI: AAH09272.1, SEQ ID NO: 16) was cloned into a pcDNA3.1 vector, and a plasmid was prepared. After the plasmid transfection (Lipofectamine® 3000 Transfection Kit, purchased from Invitrogen, Cat. No. L3000-015) of an HEK293T cell line (purchased from ATCC), selective culture was performed in DMEM medium containing 5 μg/mL of puromycin and 10% (w/w) of fetal bovine serum for 2 weeks, and positive monoclonal cells were sorted into 96 well plates on a flow cytometer FACSACriaII (purchased from BD Biosciences) using the rabbit anti-human MSLN antibody (Tab020) and a goat anti-rabbit IgG Fab antibody (cell signaling, Cat. No. 4414S) and cultured under the conditions of 37° C. and 5% (v/v) CO₂. After about 2 weeks, some of the monoclonal wells were selected for amplification. The amplified clones were examined and analyzed by an FACS flow cytometer using the Tab020 antibody, and the cell strains with better growth and higher fluorescence intensity were selected for continuous expansion and cryopreserved in liquid nitrogen. The results of expression levels are shown in Table 11 and FIG. 8, showing that HEK293T-human MSLN screened with puromycin under pressure has a single positive peak, and B8, 2A4 and 2A7 are recombinant HEK293T cell strains highly expressing the human MSLN proteins, and can be used for the detection of the binding activity of antibodies to the human MSLN proteins by FACS.

TABLE 11
FACS Result of HEK293T Recombinant Cell Lines
Expressing Human MSLN Full-length Protein

| | | Mean Fluorescence Intensity of Cells | |
|---|---|---|---|
| | Clone No. of Stably | Secondary | Tab020 |
| No. | Transfected Cell Line | Antibody Control | Antibody |
| 1 | HEK293T-hMSLN-B8 | 1 | 24400 |
| 2 | HEK293T-hMSLN-2A4 | 1 | 15400 |
| 3 | HEK293T-hMSLN-2A7 | 1 | 6581 |

(G) Preparation of Recombinant HEK293T Cell Strains Expressing Chimeric Human MSLN-R3 Protein and Chicken MSLN-R1-2

In order to ensure the spatial conformation of the protein and only reserve the binding domain of the human MSLN-R3 protein, human MSLN-R1-R2 were replaced by chicken MSLN-R1-R2 with much less homology to human. A nucleotide sequence encoding the amino acid sequence (NCBI: Met487-Ser 606 of AAH09272.1 (SEQ ID NO: 16)) of human MSLN-R3 and a nucleotide sequence encoding the amino acid sequence (Gln 327-Asp514 of XP_004945280.1) of chicken MSLN-R1-2 were cloned into pcDNA3.1 vectors, and plasmids were prepared. After the plasmid transfection (Lipofectamine® 3000 Transfection Kit, purchased from Invitrogen, Cat. No. L3000-015) of an HEK293T cell line (purchased from ATCC), selective culture was performed in DMEM medium containing 5 μg/mL of puromycin and 10% (w/w) of fetal bovine serum for 2 weeks, and positive monoclonal cells were sorted into 96 well plates on a flow cytometer FACSACriaII (purchased from BD Biosciences) using the anti-human MSLN-R3 antibody (Tab106) and a goat anti-human IgG (H+L) antibody (Jackson Cat. No. 109605088) and cultured under the conditions of 37° C. and 5% (v/v) CO₂. After about 2 weeks, some of the monoclonal wells were selected for amplification. The amplified clones were examined and analyzed by an FACS flow cytometer using the Tab106 antibody, and the cell strains with better growth and higher fluorescence intensity were selected for continuous expansion and cryopreserved in liquid nitrogen. The results of expression levels are shown in Table 12 and FIG. 9, showing that HEK293T-human MSLN R3/chicken R1-2 screened with puromycin under pressure has a single positive peak, and A5, B1 and A8 are recombinant HEK293T cell strains highly expressing the human MSLN R3/chicken R1-2 proteins, and can be used for the detection of the binding activity of antibodies to the human MSLN-R3 protein by FACS.

TABLE 12
FACS Result of HEK293T Recombinant Cell Lines
Expressing Human MSLN R3/Chicken R1-2

| | | Mean Fluorescence | |
|---|---|---|---|
| | | Intensity of Cells | |
| | Clone No. of Stably | Secondary | Tab106 |
| No. | Transfected Cell Line | Antibody Control | Antibody |
| 1 | HEK 293T-human MSLN | 7 | 17600 |
| | R3/chicken R1-2-A5 | | |
| 2 | HEK 293T-human MSLN | 7 | 11800 |
| | R3/chicken R1-2-B1 | | |
| 3 | HEK 293T-human MSLN | 7 | 9766 |
| | R3/chicken R1-2-A8 | | |

(H) Preparation of Recombinant HEK293T Cell Strains Expressing Human MSLN-R3 Protein

A nucleotide sequence encoding the amino acid sequence (NCBI: Met487-Ser 606 of AAH09272.1 (SEQ ID NO: 16)) of human MSLN-R3 was cloned into a pcDNA3.1 vector, and a plasmid was prepared. After the plasmid transfection (Lipofectamine® 3000 Transfection Kit, purchased from Invitrogen, Cat. No. L3000-015) of an HEK293T cell line (purchased from ATCC), selective culture was performed in DMEM medium containing 5 μg/mL of puromycin and 10% (w/w) of fetal bovine serum for 2 weeks, and positive monoclonal cells were sorted into 96 well plates on a flow cytometer FACSACriaII (purchased from BD Biosciences) using the anti-human MSLN-R3 antibody (Tab106) and a goat anti-human IgG (H+L) antibody (Jackson Cat. No. 109605088) and cultured under the conditions of 37° C. and 5% (v/v) CO₂. After about 2 weeks, some of the monoclonal wells were selected for amplification. The amplified clones were examined and analyzed by an FACS flow cytometer using the Tab106 antibody, and the cell strains with better growth and higher fluorescence intensity were selected for continuous expansion and cryopreserved in liquid nitrogen. The results of expression levels are shown in Table 13 and FIG. 10, showing that HEK293T-human MSLN-R3 screened with puromycin under pressure has a relatively single positive peak, and can be used for the detection of the binding activity of antibodies to the MSLN-R3 protein by FACS.

TABLE 13
FACS Result of HEK293T Recombinant Cell
Lines Expressing Human MSLN-R3 Protein

| | | Mean Fluorescence Intensity of Cells | |
|---|---|---|---|
| | Clone No. of Stably | Secondary | Tab 106 |
| No. | Transfected Cell Line | Antibody Control | Antibody |
| 1 | HEK293T-hMSLN R3 | 100 | 1689 |

(I) Study of Binding of Recombinant Cell Lines to Control Antibodies

The binding activities of the control antibodies to the cells expressing human MSLN or monkey MSLN are shown in Tables 14 to 16 and FIG. 11, and the IgG subtype control is human IgG1. Tab142, Tab020, Tab106 and Tab107 have good binding activity to OVCAR3 tumor cells expressing the human MSLN proteins and CHO-K1-hMSLN-2C8 recombinant cells, and the binding activity of Tab131 is relatively weak. Tab142, Tab106 and Tab107 have binding activity to HEK293T-monkey-MSLN recombinant cells, and the cross-binding activity of Tab020 and Tab131 to monkey MSLN was hardly detected under the same experimental conditions.

TABLE 14
Binding Reaction of Control Antibodies with
OVCAR3 Tumor Cells Detected by FACS

| Concentra- | Antibody | | | | | |
|---|---|---|---|---|---|---|
| tion (nM) | Tab020 | Tab106 | Tab107 | Tab131 | Tab142 | hIgG1 |
| 100 | 5643 | 6202 | 3154 | 328 | 3053 | 93 |
| 20 | 5534 | 3484 | 2636 | 211 | 2096 | 78 |
| 4 | 4407 | 2082 | 1520 | 113 | 1009 | 74 |
| 0.8 | 1716 | 858 | 619 | 82 | 389 | 76 |
| 0.16 | 533 | 308 | 229 | 76 | 155 | 74 |
| 0.032 | 222 | 140 | 111 | 75 | 99 | 75 |
| 0.0064 | 113 | 91 | 80 | 71 | 81 | 74 |
| 0.00128 | 91 | 111 | 76 | 75 | 79 | 75 |

TABLE 15
Binding Reaction of Control Antibodies with CHO-
K1-hMSLN-2C8 Recombinant Cells Detected by FACS

| Concentra- | Antibody | | | | | |
|---|---|---|---|---|---|---|
| tion (nM) | Tab020 | Tab106 | Tab107 | Tab131 | Tab142 | hIgG1 |
| 100 | 15258 | 6716 | 7862 | 4764 | 6315 | 72 |
| 20 | 15440 | 6269 | 6930 | 4650 | 6819 | 156 |
| 4 | 9001 | 2785 | 2960 | 1949 | 5157 | 110 |
| 0.8 | 2290 | 812 | 767 | 559 | 1310 | 80 |
| 0.16 | 593 | 270 | 264 | 200 | 410 | 59 |
| 0.032 | 206 | 119 | 120 | 154 | 152 | 59 |
| 0.0064 | 96 | 77 | 70 | 118 | 99 | 65 |
| 0.00128 | 75 | 68 | 63 | 62 | 71 | 116 |

TABLE 16
Binding Reaction of Control Antibodies with HEK293T-
monkey-MSLN Recombinant Cells Detected by FACS

| Concentra- | Antibody | | | | | |
|---|---|---|---|---|---|---|
| tion (nM) | Tab020 | Tab106 | Tab107 | Tab131 | Tab142 | hIgG1 |
| 100 | 126 | 3292 | 3930 | 87 | 3724 | 127 |
| 20 | 100 | 2665 | 3649 | 85 | 3654 | 84 |
| 4 | 90 | 2441 | 3140 | 86 | 3609 | 85 |
| 0.8 | 88 | 860 | 918 | 87 | 1678 | 87 |
| 0.16 | 86 | 313 | 288 | 83 | 503 | 85 |
| 0.032 | 85 | 142 | 131 | 106 | 192 | 125 |
| 0.0064 | 85 | 100 | 96 | 85 | 115 | 85 |
| 0.00128 | 85 | 90 | 119 | 93 | 98 | 84 |

### Example 2: Preparation of Single-domain Antibodies VHH for MSLN

(A) Alpaca Immunization and Serum Titer Detection

Two alpacas (Alpaca, No. NB148 and No. NB149) were immunized with a human MSLN (Glu296-Gly580) -Fc protein (purchased from Acro, Cat. No. MSN-H5253). At the time of primary immunization, the human MSLN-Fc protein was emulsified with Freund's complete adjuvant and then subcutaneously injected in multiple spots, i.e., 500 μg of human MSLN-Fc protein per alpaca. At the time of booster immunization, the human MSLN-Fc protein was emulsified with Freund's incomplete adjuvant and then subcutaneously injected in multiple spots, i.e., 250 μg of human MSLN-Fc protein per alpaca. The primary immunization and the first booster immunization were at an interval of 3 weeks, and the subsequent booster immunizations were at intervals of 3 weeks. Blood was collected one week after each booster immunization, and serums were tested for antibody titer and specificity of human MSLN-Fc by ELISA and FACS. The results are shown in FIG. 12 and Tables 17 to 20. The results indicate that the serums of the alpacas immunized by the human MSLN-Fc protein have binding activities to the hMSLN-FL-his protein, the hMSLN-R3-his protein, the hMSLN-R3-3 (purchased from GL Biochem, Cat No. 406676, Va1539-Va1588) polypeptide and Hela cells, where the highest dilution of ELISA for the hMSLN-FL-his protein is 8100 to 24300. The blank control is 1% (w/w) BSA (FIG. 12: abscissa 0), where the batches refer to alpaca serums at day 7 after the third (TB2) and fourth (TB3) booster immunizations, and the data in the table are OD450 nm values.

TABLE 17
Titers in Alpaca Serums Against hMSLN-
FL-his Protein Determined by ELISA
OD450 nm

| | Batch | | | |
|---|---|---|---|---|
| | NB148 | NB148 | NB149 | NB149 |
| Serum Dilution | (TB2) | (TB3) | (TB2) | (TB3) |
| 1:100 | 2.56 | 2.07 | 1.90 | 2.30 |
| 1:300 | 2.33 | 1.75 | 1.99 | 2.21 |
| 1:900 | 1.80 | 1.18 | 1.82 | 2.10 |
| 1:2700 | 0.68 | 0.48 | 1.35 | 1.78 |
| 1:8100 | 0.22 | 0.19 | 0.74 | 0.92 |
| 1:24300 | 0.11 | 0.08 | 0.27 | 0.33 |
| 1:72900 | 0.08 | 0.07 | 0.10 | 0.09 |
| 1:218700 | 0.07 | 0.06 | 0.08 | 0.08 |
| 1:656100 | 0.06 | 0.06 | 0.07 | 0.06 |
| 1:1968300 | 0.06 | 0.06 | 0.06 | 0.06 |
| 1:5904900 | 0.07 | 0.06 | 0.06 | 0.06 |
| Blank Control | 0.06 | 0.07 | 0.06 | 0.06 |

TABLE 18
Titers in Alpaca Serums Against hMSLN-
R3-his Protein Determined by ELISA
OD450 nm

| | Batch | | | |
|---|---|---|---|---|
| Serum | NB148 | NB148 | NB149 | NB149 |
| Dilution | (TB2) | (TB3) | (TB2) | (TB3) |
| 1:100 | 2.48 | 1.97 | 2.37 | 2.52 |
| 1:300 | 1.76 | 1.16 | 2.10 | 2.25 |
| 1:900 | 0.82 | 0.53 | 1.24 | 1.13 |
| 1:2700 | 0.27 | 0.16 | 0.56 | 0.51 |
| 1:8100 | 0.11 | 0.09 | 0.11 | 0.19 |
| 1:24300 | 0.08 | 0.07 | 0.08 | 0.09 |
| 1:72900 | 0.08 | 0.06 | 0.06 | 0.07 |
| 1:218700 | 0.07 | 0.06 | 0.06 | 0.06 |
| 1:656100 | 0.07 | 0.06 | 0.06 | 0.06 |
| 1:1968300 | 0.08 | 0.06 | 0.06 | 0.06 |
| 1:5904900 | 0.07 | 0.06 | 0.06 | 0.07 |
| Blank Control | 0.206* | 0.07 | 0.07 | 0.07 |

TABLE 19
Titers in Alpaca Serums Against hMSLN-
R3-3 Polypeptide Determined by ELISA
OD450 nm

| | Batch | | | |
|---|---|---|---|---|
| Serum | NB148 | NB148 | NB149 | NB149 |
| Dilution | (TB2) | (TB3) | (TB2) | (TB3) |
| 1:100 | 1.22 | 1.15 | 2.19 | 2.29 |
| 1:300 | 0.67 | 0.61 | 1.63 | 1.76 |
| 1:900 | 0.28 | 0.23 | 0.75 | 0.64 |
| 1:2700 | 0.13 | 0.12 | 0.24 | 0.21 |
| 1:8100 | 0.09 | 0.08 | 0.11 | 0.10 |
| 1:24300 | 0.07 | 0.07 | 0.08 | 0.08 |
| 1:72900 | 0.07 | 0.07 | 0.07 | 0.06 |
| 1:218700 | 0.07 | 0.07 | 0.07 | 0.06 |
| 1:656100 | 0.08 | 0.08 | 0.06 | 0.06 |
| 1:1968300 | 0.07 | 0.07 | 0.07 | 0.06 |
| 1:5904900 | 0.08 | 0.07 | 0.06 | 0.07 |
| Blank Control | 0.08 | 0.07 | 0.07 | 0.07 |

TABLE 20
Titters in Alpaca Serums Immunized with Human
MSLN-Fc Protein Against Hela Cells Detected by FACS
Mean Fluorescence Intensity MFI

| | Batch | | | |
|---|---|---|---|---|
| | NB148 | NB148 | NB149 | NB149 |
| Serum Dilution | (TB2) | (TB3) | (TB2) | (TB3) |
| 1:100 | 844 | 950 | 1476 | 1614 |
| 1:1000 | 325 | 286 | 1076 | 1253 |
| 1:10000 | 132 | 110 | 271 | 338 |

(B) Construction of Phage Library and Panning for MSLN Nanobodies

One week after four protein immunizations, 100 mL of alpaca peripheral blood was collected, PBMCs were isolated using lymphocyte separation medium, and total RNA was extracted using RNAiso Plus reagent. The extracted RNA was reversely transcribed into cDNA using PrimeScript™ II 1st Strand cDNA Synthesis Kit (purchased from Takara, Cat. No. 6210A). A variable region nucleic acid fragment encoding a heavy-chain antibody was amplified by nested PCR:

### First Round of PCR:

upstream primer (SEQ ID NO: 18):
CTTGGTGGTCCTGGCTGC;
downstream primer (SEQ ID NO: 19):
GGTACGTGCTGTTGAACTGTTCC;

### Second Round of PCR:

taking the product of the first round of PCR as a template,

upstream primer (SEQ ID NO: 20):
CATGCCATGACTGTGGCCCAGGC
GGCCCAGKTGCAGCTCGTGGAGTC;
downstream primer-1 (SEQ ID NO: 21):
CATGCCATGACTCGCGGCCGGCC
TGGCCATGGGGGTCTTCGCTGTGGTGCG;
downstream primer-2 (SEQ ID NO: 22):
CATGCCATGACTCGCGGCCGGCCT
GGCCGTCTTGTGGTTTTGGTGTCTTGGG.

The target single-domain antibody nucleic acid fragment was collected and cloned into the phage display vector pcomb3XSS (purchased from Chengdu NBbiolab, Co. Ltd) using the restriction enzyme SfiI (NEB, Cat. No. R0123S). The product was then electrotransformed into E. coli electroporation competent cells TG1, and a single-domain antibody phage display library for MSLN was constructed and assayed. The library capacity was calculated to be 3.08×10⁹ by gradient dilution plating. To check the library for insertion rate, 48 clones were randomly selected for colony PCR. The results show that the insertion rate reaches 100%.

(C) Screening of Single-domain Antibodies for MSLN

The human MSLN-FL-His protein was diluted with carbonate buffer with a pH value of 9.6 to a final concentration of 5 μg/mL and added into enzyme-labeled wells at 100 μL/well, each protein coating 8 wells overnight at 4° C.; the coating solution was discarded, washing was performed with PBS 3 times, and 300 μL of 3% BSA-PBS blocking buffer was added into each well for 1 hour of blocking at 37° C.; washing was performed with PBS 3 times, and 100 μL of phage library was added for 1 hour of incubation at 37° C.; unbound phages were sucked out and washed with PBST 6 times and with PBS twice; 100 μL of Gly-HCl eluent was added for 8 minutes of incubation at 37° C., and specifically bound phages were eluted; the eluent was transferred into a 1.5 mL sterile centrifuge tube and quickly neutralized with 10 μL of Tris-HCl neutralizing buffer; 10 μL of eluate was taken for gradient dilution, the titer was determined, the recovery rate of panning was calculated, and the rest eluates were mixed, amplified and purified for the next round of affinity panning.

From the plate with the titer of the eluates panned in the first round, 192 single clones were randomly picked with a sterilized toothpick, inoculated into 1 mL of 2×YT-AK and shaken to be cultured at 37° C. and 220 rpm for 8 hours. 100 μL of the aforementioned culture was taken, added with M13K07 phage according to cell: phage=1:20, kept still at 37° C. for 15 minutes and shaken to be cultured at 220 rpm for 45 minutes. 2×YT-AK in a volume of 300 μL was replenished, and violent shaking culture was performed at 30° C. overnight. The next day, centrifugation was performed at 12000 rpm for 2 minutes, and the supernatant was taken for monoclonal ELISA identification.

The human MSLN-FL-his protein was diluted with carbonate buffer with a pH value of 9.6 to a final concentration of 2 μg/mL and added into enzyme-labeled wells at 100 μL per well for coating overnight at 4° C.; the coating solution was discarded, washing was performed with PBST 3 times, and 300 μL of 5% skim milk was added into each well for 1 hour of blocking at 37° C.; washing was performed with PBST 3 times, and 50 μL of phage culture solution supernatant and 50 μL of 5% skim milk were added into each well for 1 hour of incubation at 37° C.; washing was performed with PBST 5 times, and horseradish peroxidase-labeled anti-M13 antibody (diluted with PBS according to 1:10000) was added at 100 μL/well to act at 37° C. for 1 hour; and the plate was washed with PBST 6 times. TMB color development solution was added for color development at 100 μL/well at 37° C. for 7 minutes, stop solution was added to stop the reaction at 50 μL/well, and optical density was measured at a wavelength of 450 nm. Human MSLN-FL-his positive clones were chosen and sent to Tsingke Biotechnology Co., Ltd for sequencing. The sequencing result was analyzed, a phylogenetic tree was constructed according to VHH-encoded protein sequences, the sequences with closer distance on the phylogenetic tree were eliminated according to sequence similarity, 12 clones were obtained by screening, and the CDRs of the sequences of the clones were analyzed using KABAT, Chothia or IMGT software respectively. The corresponding sequence information is shown in Table 21 below. The production and identification of VHH-hFc were then carried out.

TABLE 21
VHH Sequence Information

| Antibody Name | Sequence No. | Amino Acid Sequence | |
|---|---|---|---|
| NB148-13 | SEQ ID NO: 23 | QLQLVESGGGLVQAGGSLRLSCTASGSTSMSYGAKWFRRAPGKE | |
| | | REWVASVYRDGTTGYDDSVKGRFTISRDLAKNTVYLQMNSLRL | |
| | | EDTAVYYCTALNSWGQGTQVTVSS | |
| NB148-25 | SEQ ID NO: 24 | QVQLVESGGGLVQAGGSLRLSCAASGSTFGRIVMRWYRQAQGK | |
| | | QREWVASIYDSGSTSYSDPVQGRFTISRDNARNTVYLQMNSLRPA | |
| | | DTAVYYCNLGTALSSYWGQGTQVTVSS | |
| NB148-27 | SEQ ID NO: 25 | EVQVVESGGGLVQAGASLRLSCASSGRTISPIAMAWFRQAPGKEREL | |
| | | VAGLTWTGRTYYSDSVKGRFTISRDNAKNTVYLQMDSLKFED | |
| | | TAVYFCAAGDNPDRRGSSWSSSSVYEYWGQGTQVTVSS | |
| NB148-35 | SEQ ID NO: 26 | EVQVVESGGGLVQPGGSLRLSCGVSGSILALNTMRWYRQTPGNQ | |
| | | REWVASIYSSGGSTGYADSVKGRFIISRDSAKNMMSLQMNSLKSE | |
| | | DTAVYYCNVQTPLNVYWGQGTQVTVSS | |
| NB148-46 | SEQ ID NO: 27 | EVQLVESGGGLVQAGGSLRLSCAASGSTFSSYPMGWYRQAPGK | |
| | | QRELVAAISSGGSTNYADSVKGRFTISRDNAKNTVYLQMNSLKPE | |
| | | DTAVYYCNTRSPRVPNDSWGQGTQVTVSS | |
| NB148-88 | SEQ ID NO: 28 | QLQLVESGGGLVQPGGSHRLSCAVSVSTFTRYAMRWYRQAPGKERE | |
| | | WLTSIYSDGSSAYADSVKDRFTISLDNAKNMLYLQMNSLKPED | |
| | | TAVYYCNANVDSKAYWGQGTQVTVAS | |
| NB149-31 | SEQ ID NO: 29 | EVQVVESGGGLVQPGGSLRLSCAASGITFSGNYGGWFRQAPGEQ | |
| | | RELVASMSSALGTKYADPVKGRFTISTDTAKNTVDLQMNSLGPE | |
| | | DTAVYYCYARVRLSRGGWGSEYWGQGTQVTVSS | |
| NB149-34 | SEQ ID NO: 30 | QLQLVESGGGLVQPGGSLRLSCAASGSVNSITAMGWYRQGPGNE | |
| | | RELVAVITDRGRASYADSVKGRFAISMDNDKHTVYLQMDSLKPE | |
| | | DTAVYYCNAHRRTWPHPGDYWGQGTQVTVSS | |
| NB149-70 | SEQ ID NO: 31 | QVQLVESGGGLVQPGGSLRLSCAASGSIFSGNAVAWYRQAPGKE | |
| | | REAVAVITRDGDTKYADSVKGRFTISRYNAKNIAYLQMNSLKPED | |
| | | TAVYYCNTARGAAVDSWGQGTQVTVSS | |
| NB149-81 | SEQ ID NO: 32 | EVQLVESGGGLVQAGGALRLSCAASERTFSSSSMGWFRQAPGKE | |
| | | REFVAAINRIGGHIYYADSVKGRFTISKDNAKNTVYLQMSSLKPE | |
| | | DTAVYYCASSRIYSHSARDYDYWGQGTQVTVSS | |
| NB149-95 | SEQ ID NO: 33 | QVQLVESGGGLVQAGGSLRLSCTASESGFSANYMGWYRQEAPG | |
| | | KERELVATINRFGSTNYADSVKGRFTISRDNAKNTVYLQMNSLKS | |
| | | EDTGVYYCRIMRPGNWYWGQGTQVTVSS | |
| NB149-97 | SEQ ID NO: 34 | EVQVVESGGGLVQAGGGLRLSCAASERTFSSSSIGWFRQAPGKE | |
| | | REFVATMNRIGGHTFYADPVKGRFTISRDNAANTVYLQMSSLKPE | |
| | | DAAVYYCASSRIYSHSPRDYDYWSQGTQVTVSS | |
| Antibody | CDR1 | CDR2 | CDR3 |
| Name/No. | Sequence No. | Sequence No. | Sequence No. |

KABAT Analysis

| | | | |
|---|---|---|---|
| NB148-13 | SYGAK | SVYRDGTTGYDDSVKG | LNS |
| (SEQ ID NO: 23) | (SEQ ID NO: 35) | (SEQ ID NO: 36) | (SEQ ID NO: 37) |
| NB148-25 | RIVMR | SIYDSGSTSYSDPVQG | GTALSSY |
| (SEQ ID NO: 24) | (SEQ ID NO: 38) | (SEQ ID NO: 39) | (SEQ ID NO: 40) |
| NB148-27 | PIAMA | GLTWTGRTYYSDSVKG | GDNPDRRGSSWSSSSVYEY |
| (SEQ ID NO: 25) | (SEQ ID NO: 41) | (SEQ ID NO: 42) | (SEQ ID NO: 43) |
| NB148-35 | LNTMR | SIYSSGGSTGYADSVKG | QTPLNVY |
| (SEQ ID NO: 26) | (SEQ ID NO: 44) | (SEQ ID NO: 45) | (SEQ ID NO: 46) |
| NB148-46 | SYPMG | AISSGGSTNYADSVKG | RSPRVPNDS |
| (SEQ ID NO: 27) | (SEQ ID NO: 47) | (SEQ ID NO: 48) | (SEQ ID NO: 49) |
| NB148-88 | RYAMR | SIYSDGSSAYADSVKD | NVDSKAY |
| (SEQ ID NO: 28) | (SEQ ID NO: 50) | (SEQ ID NO: 51) | (SEQ ID NO: 52) |
| NB149-31 | GNYGG | SMSSALGTKYADPVKG | RVRLSRGGWGSEY |
| (SEQ ID NO: 29) | (SEQ ID NO: 53) | (SEQ ID NO: 54) | (SEQ ID NO: 55) |
| NB149-34 | ITAMG | VITDRGRASYADSVKG | HRRTWPHPGDY |
| (SEQ ID NO: 30) | (SEQ ID NO: 56) | (SEQ ID NO: 57) | (SEQ ID NO: 58) |
| NB149-70 | GNAVA | VITRDGDTKYADSVKG | ARGAAVDS |
| (SEQ ID NO: 31) | (SEQ ID NO: 59) | (SEQ ID NO: 60) | (SEQ ID NO: 61) |
| NB149-81 | SSSMG | AINRIGGHIYYADSVKG | SRIYSHSARDYDY |
| (SEQ ID NO: 32) | (SEQ ID NO: 62) | (SEQ ID NO: 63) | (SEQ ID NO: 64) |
| NB149-95 | ANYMG | TINRFGSTNYADSVKG | MRPGNWY |
| (SEQ ID NO: 33) | (SEQ ID NO: 65) | (SEQ ID NO: 66) | (SEQ ID NO: 67) |
| NB149-97 | SSSIG | TMNRIGGHTFYADPVKG | SRIYSHSPRDYDY |
| (SEQ ID NO: 34) | (SEQ ID NO: 68) | (SEQ ID NO: 69) | (SEQ ID NO: 70) |

Chothia Analysis

| | | | |
|---|---|---|---|
| NB148-13 | GSTSMSY | YRDGT | LNS |
| (SEQ ID NO: 23) | (SEQ ID NO: 71) | (SEQ ID NO: 72) | (SEQ ID NO: 73) |
| NB148-25 | GSTFGRI | YDSGS | GTALSSY |
| (SEQ ID NO: 24) | (SEQ ID NO: 74) | (SEQ ID NO: 75) | (SEQ ID NO: 76) |
| NB148-27 | GRTISPI | TWTGR | GDNPDRRGSSWSSSSVYEY |
| (SEQ ID NO: 25) | (SEQ ID NO: 77) | (SEQ ID NO: 78) | (SEQ ID NO: 79) |
| NB148-35 | GSILALN | YSSGGS | QTPLNVY |
| (SEQ ID NO: 26) | (SEQ ID NO: 80) | (SEQ ID NO: 81) | (SEQ ID NO: 82) |
| NB148-46 | GSTFSSY | SSGGS | RSPR VPNDS |
| (SEQ ID NO: 27) | (SEQ ID NO: 83) | (SEQ ID NO: 84) | (SEQ ID NO: 85) |
| NB148-88 | VSTFTRY | YSDGS | NVDSKAY |
| (SEQ ID NO: 28) | (SEQ ID NO: 86) | (SEQ ID NO: 87) | (SEQ ID NO: 88) |
| NB149-31 | GITFSGN | SSALG | RVRLSRGGWGSEY |
| (SEQ ID NO: 29) | (SEQ ID NO: 89) | (SEQ ID NO: 90) | (SEQ ID NO: 91) |
| NB149-34 | GSVNSIT | TDRGR | HRRTWPHPGDY |
| (SEQ ID NO: 30) | (SEQ ID NO: 92) | (SEQ ID NO: 93) | (SEQ ID NO: 94) |
| NB149-70 | GSIFSGN | TRDGD | ARGAAVDS |
| (SEQ ID NO: 31) | (SEQ ID NO: 95) | (SEQ ID NO: 96) | (SEQ ID NO: 97) |
| NB149-81 | ERTFSSS | NRIGGH | SRIYSHSARDYDY |
| (SEQ ID NO: 32) | (SEQ ID NO: 98) | (SEQ ID NO: 99) | (SEQ ID NO: 100) |
| NB149-95 | ESGFSAN | NRFGS | MRPGNWY |
| (SEQ ID NO: 33) | (SEQ ID NO: 101) | (SEQ ID NO: 102) | (SEQ ID NO: 103) |
| NB149-97 | ERTFSSS | NRIGGH | SRIYSHSPRDYDY |
| (SEQ ID NO: 34) | (SEQ ID NO: 104) | (SEQ ID NO: 105) | (SEQ ID NO: 106) |

IMGT Analysis

| | | | |
|---|---|---|---|
| NB148-13 | GSTSMSYG | VYRDGTT | TALNS |
| (SEQ ID NO: 23) | (SEQ ID NO: 107) | (SEQ ID NO: 108) | (SEQ ID NO: 109) |
| NB148-25 | GSTFGRIV | IYDSGST | NLGTALSSY |
| (SEQ ID NO: 24) | (SEQ ID NO: 110) | (SEQ ID NO: 111) | (SEQ ID NO: 112) |
| NB148-27 | GRTISPIA | LTWTGRT | AAGDNPDRRGSSWSSSSVYEY |
| (SEQ ID NO: 25) | (SEQ ID NO: 113) | (SEQ ID NO: 114) | (SEQ ID NO: 115) |
| NB148-35 | GSILALNT | IYSSGGST | NVQTPLNVY |
| (SEQ ID NO: 26) | (SEQ ID NO: 116) | (SEQ ID NO: 117) | (SEQ ID NO: 118) |
| NB148-46 | GSTFSSYP | ISSGGST | NTRSPRVPNDS |
| (SEQ ID NO: 27) | (SEQ ID NO: 119) | (SEQ ID NO: 120) | (SEQ ID NO: 121) |
| NB148-88 | VSTFTRYA | IYSDGSS | NANVDSKAY |
| (SEQ ID NO: 28) | (SEQ ID NO: 122) | (SEQ ID NO: 123) | (SEQ ID NO: 124) |
| NB149-31 | GITFSGNY | MSSALGT | YARVRLSRGGWGSEY |
| (SEQ ID NO: 29) | (SEQ ID NO: 125) | (SEQ ID NO: 126) | (SEQ ID NO: 127) |
| NB149-34 | GSVNSITA | ITDRGRA | NAHRRTWPHPGDY |
| (SEQ ID NO: 30) | (SEQ ID NO: 128) | (SEQ ID NO: 129) | (SEQ ID NO: 130) |
| NB149-70 | GSIFSGNA | ITRDGDT | NTARGAAVDS |
| (SEQ ID NO: 31) | (SEQ ID NO: 131) | (SEQ ID NO: 132) | (SEQ ID NO: 133) |
| NB149-81 | ERTFSSSS | INRIGGHI | ASSRIYSHSARDYDY |
| (SEQ ID NO: 32) | (SEQ ID NO: 134) | (SEQ ID NO: 135) | (SEQ ID NO: 136) |
| NB149-95 | ESGFSANY | INRFGST | RIMRPGNWY |
| (SEQ ID NO: 33) | (SEQ ID NO: 137) | (SEQ ID NO: 138) | (SEQ ID NO: 139) |
| NB149-97 | ERTFSSSS | MNRIGGHT | ASSRIYSHSPRDYDY |
| (SEQ ID NO: 34) | (SEQ ID NO: 140) | (SEQ ID NO: 141) | (SEQ ID NO: 142) |

### Example 3: Production of VHH-hFc

The target VHH sequence was recombined into an expression vector for human IgG1 Fc to give a recombinant plasmid. For the details of the plasmid construction, transfection and purification procedures, refer to example 1 (A), and the sequence of human IgG1 Fc is set forth in SEQ ID NO: 11.

The purified VHH-hFc was assayed and analyzed for protein concentration, purity, endotoxin (Lonza kit), etc. The results are shown in Table 22, indicating that the antibodies have high purity and an endotoxin concentration less than 1.0 EU/mg.

TABLE 22
Quality Control of VHH-hFc

| | Antibody | | |
|---|---|---|---|
| Antibody | Concentration | Antibody Purity | Endotoxin |
| Name | (mg/mL) | (SEC, 280 nm) % | EU/mg |
| NB148-13 | 2.77 | 99.02 | <1 |
| NB148-25 | 3.31 | 100.00 | <1 |
| NB148-27 | 3.85 | 98.53 | <1 |
| NB148-35 | 3.22 | 99.05 | <1 |
| NB148-46 | 2.95 | 99.71 | <1 |
| NB148-88 | 3.64 | 99.29 | <1 |
| NB149-31 | 2.09 | 98.98 | <1 |
| NB149-34 | 2.50 | 99.79 | <1 |
| NB149-70 | 3.05 | 99.27 | <1 |
| NB149-81 | 2.88 | 99.96 | <1 |
| NB149-95 | 2.33 | 100.00 | <1 |
| NB149-97 | 1.73 | 99.30 | <1 |

### Example 4: Identification of VHH-hFc Antibodies

(A) Binding of VHH-hFc to Human MSLN Proteins/Polypeptide determined by Enzyme-linked Immunosorbent Assay (ELISA)

The human MSLN-FL-his, human MSLN-R1-his, human MSLN-R2-his and human MSLN-R3-his proteins and the human MSLN-R3-3 polypeptide were diluted with PBS to a final concentration of 2 μg/mL and then added into a 96-well ELISA plate at 50 μL per well. The plate was sealed with a plastic film for incubation at 4° C. overnight. The next day, the plate was washed with PBS twice, and a blocking buffer [PBS+2% (w/w) BSA] was added for 2 hours of blocking at room temperature. The blocking buffer was decanted, and the VHH-hFc or negative control antibody with a starting concentration of 100 nM serially diluted by 10 folds was added at 50 μl per well. After 2 hours of incubation at 37° C., the plate was washed with PBS 3 times. An HRP (horseradish peroxidase) -labeled secondary antibody (purchased from Sigma, Cat. No. A0170) was added, and after 1 hour of incubation at 37° C., the plate was washed with PBS 5 times. TMB substrate was added at 50 μl per well, and after 10 minutes of incubation at room temperature, stop solution (1.0 M HCl) was added at 50 μl per well. OD450 nm values were read using an ELISA plate reader (Multimode Plate Reader, EnSight, purchased from Perkin Elmer). The results of binding activity of VHH-hFc to the human MSLN proteins/polypeptide are shown in FIGS. 13A to 13B, FIG. 14 and Tables 23 to 27, indicating that purified VHH-hFc can bind to the human MSLN-FL-his protein but cannot bind to the human MSLN-R3-his protein, NB148-27, NB148-46, NB149-31, NB149-34, NB149-70, and NB149-95 can bind to the MSLN-R1-his protein, and NB148-13, NB148-25, NB148-35 and NB148-88 can bind to the MSLN-R2-his protein, where the IgG control is hIgG1, and the data in the tables are OD450 nm values.

TABLE 23
Binding Reaction of VHH-hFc with Human
MSLN-FL-his Protein Determined by ELISA
OD450 nm

| Antibody | Concentration (nM) | | | | | | | |
|---|---|---|---|---|---|---|---|---|
| Name | 100 | 10 | 1 | 0.1 | 0.01 | 0.001 | 0.0001 | 0 |
| NB148-13 | 2.28 | 2.46 | 1.69 | 0.36 | 0.08 | 0.05 | 0.04 | 0.05 |
| NB148-25 | 2.64 | 2.62 | 1.16 | 0.19 | 0.06 | 0.05 | 0.05 | 0.05 |
| NB148-27 | 2.53 | 2.69 | 1.98 | 0.44 | 0.10 | 0.05 | 0.05 | 0.05 |
| NB148-35 | 2.56 | 2.61 | 1.82 | 0.24 | 0.06 | 0.05 | 0.05 | 0.05 |
| NB148-46 | 3.21 | 2.63 | 1.74 | 0.35 | 0.08 | 0.05 | 0.05 | 0.05 |
| NB148-88 | 2.88 | 2.67 | 1.67 | 0.27 | 0.07 | 0.05 | 0.05 | 0.05 |
| NB149-31 | 2.63 | 2.14 | 1.40 | 0.28 | 0.07 | 0.05 | 0.05 | 0.05 |
| NB149-34 | 2.62 | 2.07 | 0.86 | 0.17 | 0.06 | 0.05 | 0.05 | 0.05 |
| NB149-70 | 2.30 | 2.16 | 1.61 | 0.39 | 0.08 | 0.05 | 0.05 | 0.05 |
| NB149-81 | 2.37 | 2.09 | 1.25 | 0.22 | 0.06 | 0.05 | 0.04 | 0.05 |
| NB149-95 | 2.40 | 2.35 | 0.43 | 0.07 | 0.05 | 0.05 | 0.04 | 0.06 |
| NB149-97 | 2.60 | 2.28 | 2.29 | 1.75 | 0.45 | 0.10 | 0.06 | 0.06 |
| Tab108 | 1.17 | 1.36 | 0.89 | 0.53 | 0.17 | 0.07 | 0.05 | 0.05 |
| Tab106 | 2.83 | 2.77 | 0.81 | 0.14 | 0.05 | 0.05 | 0.05 | 0.05 |
| hIgG1 | 0.05 | 0.05 | 0.05 | 0.05 | 0.05 | 0.05 | 0.05 | 0.05 |

TABLE 24
Binding Reaction of VHH-hFc with Human
MSLN-R1-his Protein Determined by ELISA
OD450 nm

| Antibody | Concentration (nM) | | | | | | | |
|---|---|---|---|---|---|---|---|---|
| Name | 100 | 10 | 1 | 0.1 | 0.01 | 0.001 | 0.0001 | 0 |
| NB149-97 | 0.10 | 0.07 | 0.06 | 0.06 | 0.05 | 0.06 | 0.06 | 0.06 |
| Tab108 | 0.12 | 0.06 | 0.05 | 0.05 | 0.06 | 0.05 | 0.05 | 0.06 |
| hIgG1 | 0.20 | 0.07 | 0.05 | 0.05 | 0.05 | 0.06 | 0.05 | 0.06 |

TABLE 25
Binding Reaction of VHH-hFc with Human
MSLN-R2-his Protein Determined by ELISA
OD450 nm

| Antibody | Concentration (nM) | | | | | | | |
|---|---|---|---|---|---|---|---|---|
| Name | 100 | 10 | 1 | 0.1 | 0.01 | 0.001 | 0.0001 | 0 |
| NB149-97 | 0.08 | 0.06 | 0.05 | 0.05 | 0.05 | 0.05 | 0.05 | 0.06 |
| Tab108 | 0.10 | 0.06 | 0.05 | 0.05 | 0.05 | 0.05 | 0.05 | 0.06 |
| hIgG1 | 0.16 | 0.06 | 0.05 | 0.05 | 0.05 | 0.05 | 0.05 | 0.05 |

TABLE 26
Binding Reaction of VHH-hFc with Human
MSLN-R3-his Protein Determined by ELISA
OD450 nm

| Antibody | Concentration (nM) | | | | | | | |
|---|---|---|---|---|---|---|---|---|
| Name | 100 | 10 | 1 | 0.1 | 0.01 | 0.001 | 0.0001 | 0 |
| NB148-13 | 0.05 | 0.05 | 0.05 | 0.05 | 0.05 | 0.05 | 0.05 | 0.05 |
| NB148-25 | 0.06 | 0.05 | 0.05 | 0.05 | 0.05 | 0.05 | 0.05 | 0.05 |
| NB148-27 | 0.05 | 0.05 | 0.05 | 0.05 | 0.05 | 0.05 | 0.05 | 0.05 |
| NB148-35 | 0.05 | 0.05 | 0.05 | 0.05 | 0.05 | 0.05 | 0.05 | 0.05 |
| NB148-46 | 0.05 | 0.05 | 0.05 | 0.05 | 0.05 | 0.05 | 0.05 | 0.05 |
| NB148-88 | 0.05 | 0.05 | 0.05 | 0.05 | 0.05 | 0.05 | 0.05 | 0.05 |
| NB149-31 | 0.05 | 0.05 | 0.05 | 0.05 | 0.05 | 0.05 | 0.05 | 0.05 |
| NB149-34 | 0.07 | 0.05 | 0.05 | 0.18 | 0.06 | 0.05 | 0.05 | 0.05 |
| NB149-70 | 0.05 | 0.05 | 0.05 | 0.05 | 0.05 | 0.05 | 0.05 | 0.05 |
| NB149-81 | 0.06 | 0.05 | 0.04 | 0.05 | 0.05 | 0.05 | 0.05 | 0.05 |
| NB149-95 | 0.06 | 0.05 | 0.05 | 0.05 | 0.05 | 0.05 | 0.05 | 0.05 |
| NB149-97 | 0.09 | 0.06 | 0.06 | 0.06 | 0.06 | 0.06 | 0.06 | 0.06 |
| Tab108 | 0.74 | 0.65 | 0.25 | 0.08 | 0.06 | 0.05 | 0.05 | 0.06 |
| Tab106 | 1.81 | 1.04 | 0.22 | 0.07 | 0.05 | 0.05 | 0.05 | 0.05 |
| hIgG1 | 0.06 | 0.05 | 0.05 | 0.05 | 0.05 | 0.05 | 0.05 | 0.05 |

TABLE 27
Binding Reaction of VHH-hFc with Human MSLN
Proteins (FIGS. 13A and 13B) Determined by ELISA
OD450 nm

| | Concentration (nM) | | | | | | | |
|---|---|---|---|---|---|---|---|---|
| | hMSLN-FL-his | | hMSLN-R1-his | | hMSLN-R2-his | | hMSLN-R3-his | |
| Antibody | 20 | 0.2 | 20 | 0.2 | 20 | 0.2 | 20 | 0.2 |
| NB148-13 | 2.63 | 2.36 | 0.31 | 0.12 | 2.33 | 2.17 | 0.06 | 0.05 |
| NB148-25 | 2.71 | 2.35 | 0.46 | 0.14 | 2.31 | 1.92 | 0.18 | 0.06 |
| NB148-27 | 2.75 | 2.39 | 2.00 | 1.13 | 0.12 | 0.06 | 0.07 | 0.05 |
| NB148-35 | 2.87 | 2.42 | 0.14 | 0.06 | 2.68 | 2.27 | 0.11 | 0.05 |
| NB148-46 | 2.62 | 2.40 | 1.69 | 1.05 | 0.17 | 0.07 | 0.05 | 0.05 |
| NB148-88 | 2.56 | 2.40 | 0.25 | 0.19 | 2.45 | 2.18 | 0.06 | 0.05 |
| NB149-31 | 2.74 | 2.58 | 2.05 | 1.70 | 0.14 | 0.08 | 0.06 | 0.05 |
| NB149-34 | 2.72 | 2.48 | 0.78 | 0.19 | 0.08 | 0.07 | 0.06 | 0.05 |
| NB149-70 | 2.65 | 2.48 | 2.25 | 1.79 | 0.14 | 0.09 | 0.05 | 0.05 |
| NB149-81 | 2.54 | 2.31 | 0.06 | 0.05 | 0.12 | 0.08 | 0.05 | 0.05 |
| NB149-95 | 2.66 | 2.47 | 2.41 | 2.02 | 0.34 | 0.10 | 0.06 | 0.05 |
| Tab142 | 2.79 | 2.46 | 2.46 | 2.16 | 0.24 | 0.10 | 0.06 | 0.05 |
| Tab131 | 0.09 | 0.08 | 0.05 | 0.05 | 0.08 | 0.07 | 0.07 | 0.05 |
| Tab106 | 2.45 | 1.95 | 0.56 | 0.08 | 1.44 | 0.13 | 1.89 | 0.87 |
| hIgG1 | 0.28 | 0.24 | 0.07 | 0.06 | 0.09 | 0.08 | 0.08 | 0.06 |

(B) Binding of VHH-hFc to Recombinant Cells Expressing Human MSLN Proteins by Flow Cytometry Assay (FACS)

The desired cells were expanded to the logarithmic growth phase in a T-175 cell culture flask, the medium was sucked off, washing was performed with PBS buffer twice, the cells were trypsinized, the trypsinization was then stopped with complete medium, and the cells were blown up to a single-cell suspension. After cell counting, centrifugation was performed, the cell pellet was resuspended to 2×10⁶ cells per ml with FACS buffer (PBS+2% fetal bovine serum), the cell suspension was added into a 96-well FACS reaction plate at 50 μl per well, and a VHH-hFc sample to be tested with an initial concentration of 200 nM gradiently diluted by 5 folds was added at 50 μl per well and uniformly mixed with the cell suspension for 1 hour of incubation at 4° C. After the plate was centrifuged and washed with PBS buffer 3 times, an FITC-labeled secondary antibody (purchased from Invitrogen, Cat. No. A18830) was added at 50 μL per well for 1 hour of incubation at 4° C. The results were tested and analyzed by FACS (FACS Canto™, purchased from BD) after the plate was centrifuged and washed with PBS buffer 3 times and resuspended with 100 μL of PBS. Data analysis was performed by software (FlowJo) to obtain the mean fluorescence intensity (MFI) of the cells. Then, analysis was performed by software (GraphPad Prism8), data were fitted, and EC50 values were calculated. The analysis results are shown in Tables 28 to 31, FIGS. 15A and 15B and FIGS. 16 to 18, indicating that VHH-hFc can specifically bind to both recombinant cells expressing the human MSLN proteins and tumor cells, but does not have binding activity to recombinant cells expressing the human MSLN-R3 protein.

TABLE 28
Binding Reactions of VHH-hFc with Cells Expressing Human
MSLN Proteins and Negative Cells Detected by FACS

| | CHO-K1-hMSLN-2C8 | | Hela | | | |
|---|---|---|---|---|---|---|
| | Maximum | | Maximum | | | |
| | Mean | | Mean | | CHO-K1 | |
| | Fluorescence | | Fluorescence | | Mean Fluorescence Intensity | |
| | Intensity | | Intensity | | MFI | |
| Antibody Name | Max MFI | Ec50(nM) | Max MFI | Ec50(nM) | 100 nM | 0.02 nM |
| NB148-13 | 13154 | 2.51 | 303 | 0.14 | 43 | 45 |
| NB148-25 | 13547 | 3.82 | 394 | 0.68 | 46 | 42 |
| NB148-27 | 10639 | 2.29 | 352 | 2.59 | 46 | 44 |
| NB148-35 | 13551 | 3.65 | 318 | 0.26 | 42 | 44 |
| NB148-46 | 10902 | 1.69 | 332 | 0.14 | 44 | 44 |
| NB148-88 | 12953 | 2.55 | 310 | 0.29 | 44 | 45 |
| NB149-31 | 11337 | 1.85 | 313 | 0.16 | 44 | 44 |
| NB149-34 | 11739 | 2.23 | 382 | 0.16 | 47 | 44 |
| NB149-70 | 11338 | 2.22 | 303 | 0.13 | 46 | 45 |
| NB149-81 | 11714 | 2.20 | 348 | 0.30 | 44 | 43 |
| NB149-95 | 12826 | 2.05 | 388 | 0.12 | 45 | 55 |
| Tab106 | 11714 | 4.90 | 510 | Fitted | 160 | 44 |
| | | | | difference | | |
| hIgG1 | 80 | No binding | 75 | No binding | 101 | 43 |

TABLE 29
Binding Reaction of VHH-hFc with Cells Expressing Human MSLN Proteins (FIG. 16) Detected by FACS

| | | | | | HEK293T-chickR1-2/hR | | |
|---|---|---|---|---|---|---|---|
| | OVCAR3 | | HEK293T-hMSLN-B8 | | 3-A5 | | HEK293T |
| | Maximum | | Maximum | | Maximum | | Maximum |
| | Mean | | Mean | | Mean | | Mean |
| | Fluorescence | | Fluorescence | | Fluorescence | | Fluorescence |
| Antibody | Intensity | | Intensity | | Intensity | | Intensity |
| Name | Max MFI | Ec50(nM) | Max MFI | Ec50(nM) | Max MFI | Ec50(nM) | Max MFI |
| NB149-97 | 3788 | 3.54 | 18421 | 4.00 | 66 | No binding | 94 |
| Tab108 | 4031 | 0.58 | 18978 | ~4.24 | 15546 | ~4.62 | 93 |
| hIgG1 | 147 | No binding | 77 | No binding | 115 | No binding | 92 |

TABLE 30
Binding Reaction of VHH-hFc with
Recombinant Cells Expressing Human
MSLN Proteins Detected by FACS
Mean Fluorescence Intensity MFI

| | Concentration (nM) | | | |
|---|---|---|---|---|
| | HEK293T- | | HEK293T-hR3/ | |
| | hMSLN-B8 | | chickR1-2-A5 | |
| Antibody | 20 nM | 0.2 nM | 20 nM | 0.2 nM |
| NB148-13 | 30492 | 1365 | 74 | 74 |
| NB148-25 | 29433 | 784 | 73 | 73 |
| NB148-27 | 24847 | 1670 | 93 | 73 |
| NB148-35 | 29745 | 1047 | 73 | 73 |
| NB148-46 | 25746 | 1275 | 73 | 72 |
| NB148-88 | 30587 | 1247 | 73 | 73 |
| NB149-31 | 24844 | 1210 | 74 | 74 |
| NB149-34 | 23482 | 847 | 73 | 73 |
| NB149-70 | 22834 | 1429 | 73 | 91 |
| NB149-81 | 23380 | 1331 | 74 | 71 |
| NB149-95 | 28007 | 678 | 95 | 76 |
| Tab 142 | 25123 | 1077 | 73 | 118 |
| Tab131 | 22556 | 532 | 15654 | 471 |
| Tab106 | 21923 | 1085 | 17897 | 767 |
| hIgG1 | 68 | 65 | 74 | 73 |

TABLE 31
Binding Reaction of VHH-hFc with Tumor Cells
Expressing Human MSLN Proteins Detected by FACS
Mean Fluorescence Intensity MFI

| | Concentration (nM) | | | | | |
|---|---|---|---|---|---|---|
| | OVCAR3 | | | A431 | | |
| Antibody | 20 | 2 | 0.2 | 20 | 2 | 0.2 |
| 148-27 | 5403 | 1601 | 361 | 102 | 96 | 89 |
| 148-46 | 11100 | 7152 | 1449 | 97 | 92 | 91 |
| 149-31 | 11350 | 6093 | 1201 | 94 | 90 | 89 |
| 149-34 | 12193 | 7152 | 1370 | 130 | 90 | 99 |
| 149-70 | 11437 | 6437 | 1240 | 112 | 90 | 90 |
| 149-95 | 12846 | 8928 | 1953 | 106 | 92 | 90 |
| 149-13 | 9045 | 8462 | 2705 | 112 | 92 | 90 |
| 148-25 | 8541 | 7741 | 1970 | 157 | 125 | 98 |
| 148-35 | 9222 | 7480 | 1613 | 100 | 93 | 90 |
| 148-88 | 9366 | 7140 | 1480 | 96 | 92 | 91 |
| 149-81 | 10851 | 4639 | 883 | 100 | 93 | 92 |
| 149-97 | 11628 | 5763 | 1196 | 98 | 92 | 92 |
| Tab142 | 16068 | 7155 | 1529 | 100 | 94 | 91 |
| Tab106 | 15685 | 8763 | 1894 | 953 | 201 | 101 |
| hIgG1 | 329 | 255 | 106 | 114 | 98 | 90 |

### Example 5: Detection of Cross-binding Activity of VHH-hFc

HEK293T-monkey MSLN recombinant cells were subjected to FACS detection and data analysis according to the method in Example 4 (B). The analysis results are shown in Tables 32 and 33 and FIG. 19, and the VHH-hFc antibodies NB148-27, NB148-46, NB149-31, NB149-34, NB149-70 and NB149-95 have better specific binding activity to the HEK293T-monkey MSLN cells, NB149-81 and NB149-97 have weak binding activity to the HEK293T-monkey MSLN cells, and NB148-13, NB148-25, NB148-35 and NB148-88 have no binding activity to the HEK293T-monkey-MSLN cells under the experimental conditions.

TABLE 32
Binding Reaction of VHH-hFc with
Cells Expressing Monkey MSLN
Proteins Detected by FACS

| | HEK293T-monkey MSLN | | HEK293T | |
|---|---|---|---|---|
| | Maximum Mean | | Mean Fluorescence | |
| Antibody | Fluorescence Intensity | | Intensity MFI | |
| Name | Max MFI | Ec50(nM) | 100 nM | 0.02 nM |
| NB148-13 | 106 | No binding | 47 | 38 |
| NB148-25 | 78 | No binding | 43 | 47 |
| NB148-27 | 16703 | 1.57 | 40 | 41 |
| NB148-35 | 89 | No binding | 41 | 44 |
| NB148-46 | 15596 | ~0.88 | 96 | 42 |
| NB148-88 | 136 | No binding | 48 | 40 |
| NB149-31 | 16610 | ~0.89 | 41 | 44 |
| NB149-34 | 16001 | ~0.88 | 58 | 101 |
| NB149-70 | 16861 | ~0.91 | 48 | 44 |
| NB149-81 | 5632 | Weak binding | 46 | 43 |
| NB149-95 | 20115 | 1.96 | 45 | 44 |
| Tab106 | 8645 | 1.15 | 294 | 36 |
| hIgG1 | 104 | No binding | 38 | 37 |

TABLE 33
Binding Reaction of VHH-hFc with Cells Expressing
Monkey MSLN Proteins Detected by FACS

| | HEK293T-monkey MSLN | | HEK293T | |
|---|---|---|---|---|
| | Maximum Mean | | Mean Fluorescence | |
| Antibody | Fluorescence Intensity | | Intensity MFI | |
| Name | Max MFI | Ec50(nM) | 20 nM | 0.16 nM |
| NB149-97 | 1522 | Weak binding | 94 | 93 |
| Tab108 | 4243 | ~0.83 | 93 | 93 |
| hIgG1 | 137 | No binding | 92 | 92 |

### Example 6: Detection of Affinity of VHH-hFc

(A) Detection of Affinity of VHH-hFc for Human MSLN Proteins

The anti-human MSLN VHH-hFc was captured using a Protein A chip (GE Healthcare; 29-127-558). The sample and running buffer was HBS-EP+(10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then a single concentration of human MSLN-FL-his protein was injected. The association and dissociation processes of the antibody with the antigen protein were recorded, and finally, the chip was regenerated using Glycine pH 1.5 (GE Healthcare; BR-1003-54). The association was determined by injecting different concentrations of human MSLN-FL-his in solution for 240 s on end, where the flow rate was 30 μL/min, and the protein was diluted in a 1:1 dilution ratio from 200 nM (see detailed results for actual concentrations tested) to obtain 5 concentrations in total. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections were also subtracted (=double reference). To calculate the apparent KD and other kinetic parameters, the Langmuir 1:1 model was used. The association rate (Ka), dissociation rate (Kd) and binding affinity (KD) of VHH-hFc to the human MSLN-FL-his protein are shown in Table 34 and FIG. 20, where the antibodies Tab108, Tab142 are used as controls. The results indicate that the affinity of the VHH-hFc for the human MSLN proteins is not less than 1.08E-08M.

TABLE 34
Binding Affinity of VHH-hFc for Human MSLN-FL-his Protein

| Antibody Name | Ka (1/Ms) | Kd (1/s) | KD (M) |
|---|---|---|---|
| NB148-13 | 4.46E+05 | 4.81E−03 | 1.08E−08 |
| NB148-25 | 2.24E+05 | 3.58E−04 | 1.59E−09 |
| NB148-27 | 1.44E+05 | 1.36E−03 | 9.39E−09 |
| NB148-35 | 2.47E+05 | 1.31E−03 | 5.31E−09 |
| NB148-46 | 9.02E+05 | 3.00E−03 | 3.33E−09 |
| NB148-88 | 4.99E+05 | 1.23E−03 | 2.45E−09 |
| NB149-31 | 1.30E+06 | 1.12E−03 | 8.61E−10 |
| NB149-34 | 2.18E+06 | 4.71E−04 | 2.16E−10 |
| NB149-70 | 1.46E+06 | 1.16E−03 | 7.95E−10 |
| NB149-81 | 1.09E+06 | 5.79E−04 | 5.33E−10 |
| NB149-95 | 1.39E+06 | 3.24E−05 | 2.34E−11 |
| NB149-97 | 9.45E+05 | 1.74E−04 | 1.84E−10 |
| Tab108 | 4.71E+05 | 1.21E−04 | 2.57E−10 |
| Tab142 | 1.03E+06 | 1.96E−04 | 1.90E−10 |

### Example 7: Antibody-antigen Binding Epitope Competition Experiment (Epitope Binning)

(A) Competitive ELISA Method

To identify a binding site of an antibody to an antigen, MSLN VHH-hFc was grouped using a competitive ELISA method. Referring to the method of Example 4 (A), 2 μg/mL VHH-hFc-coated ELISA plates were used, a human MSLN protein was gradiently diluted from 30 μg/mL, and EC80 was calculated as a concentration in competitive ELISA.

VHH-hFc was diluted to 2 μg/mL with PBS, and coated 96-well high-adsorption ELISA plates at 50 μL/well overnight at 4° C., 250 μL of blocking buffer (PBS containing 2% (w/w) BSA) was added for two hours of blocking at room temperature, 40 μg/mL of antibody to be tested was added, the human MSLN-FL-His protein with a concentration of EC80 corresponding to each antibody to be tested was then added for 2 hours of incubation, washing was performed with PBS 5 times, an HRP-labeled anti-His secondary antibody (purchased from Genescript, Cat. No. A00612) was then added for 1 hour of incubation, and the plates were washed 5 times. TMB substrate was added at 50 μl per well, and after 10 minutes of incubation at room temperature, stop solution (1.0 M HCl) was added at 50 μl per well. OD450 nm values were read using an ELISA plate reader (Insight, purchased from PerkinElmer), and the competition rate between the antibodies was calculated according to the OD450 nm values using a formula. The results are shown in FIG. 21, the higher the value of the competition rate, the closer the epitopes to which two antibodies bind.

(B) Competitive FACS Method

To verify a binding site of an antibody to an antigen, MSLN VHH-hFc was grouped using a competitive FACS method. Referring to the cell treatment and plating method in Example 4 (B), the binding of Biotin-Tab142 and Biotin-Tab131 to CHO-K1-human MSLN-2C8 cells was first explored, and EC80 was calculated as a concentration in the FACS competition experiment.

A VHH-hFc sample to be tested with an initial concentration of 200 nM or 400 nM gradiently diluted by 5 folds was prepared and added at 50 μl per well, 20 nM or 10 nM Biotin-Tab142 and 20 nM Biotin-Tab131 were prepared and added at 50 μl per well, and the cells were rapidly uniformly mixed and incubated for 1 hour at 4° C. After the plate was centrifuged and washed with PBS buffer 3 times, an Alexa 488-labeled secondary antibody (purchased from Invitrogen, Cat. No. S11223) was added at 50 μL per well for 1 hour of incubation at 4° C. The results were tested and analyzed by FACS (FACS Canto™, purchased from BD) after the plate was centrifuged and washed with PBS buffer 3 times and resuspended with 100 μL of PBS. Data analysis was performed by software (FlowJo) to obtain the mean fluorescence intensity (MFI) of the cells. Then, analysis was performed by software (GraphPad Prism8), and data were fitted to draw a curve. The results are shown in Tables 35 to 37 and FIGS. 22A and 22B.

The VHH-hFc was classified according to the results of the above two methods. The results are shown in FIG. 23, indicating that NB148-13, NB148-25, NB148-35, NB148-88 and Tab020 have competitive relationship with Tab131 and that NB148-46, NB149-27, NB149-31, NB149-34, NB149-70, NB149-81, NB149-95 and NB149-97 compete with Tab142 (Amatuximab, epitope R1).

TABLE 35
Results of Competition between VHH-hFc and Biotin-Tab142

| Competitive | Concentration (nM) | | | | | | | |
|---|---|---|---|---|---|---|---|---|
| Antibodies | 100 | 20 | 4 | 0.8 | 0.16 | 0.032 | 0.0064 | 0.00128 |
| NB148-25 | 40116 | 41561 | 41111 | 41785 | 41529 | 41608 | 42202 | 42112 |
| NB148-27 | 26032 | 33102 | 40393 | 41657 | 40478 | 41726 | 41657 | 40960 |
| NB148-35 | 40559 | 41135 | 41160 | 43223 | 42011 | 42868 | 39872 | 41012 |
| NB148-46 | 19540 | 24947 | 33539 | 39252 | 41041 | 43518 | 43784 | 42611 |
| NB148-88 | 45166 | 42588 | 43010 | 42005 | 43199 | 44671 | 41716 | 41079 |
| NB149-31 | 17558 | 25591 | 33598 | 39823 | 41018 | 42736 | 43493 | 40494 |
| NB149-34 | 17194 | 24639 | 31458 | 39728 | 41651 | 43112 | 42530 | 40762 |
| NB149-70 | 20199 | 25096 | 33357 | 40383 | 41015 | 42743 | 42146 | 40643 |
| NB149-81 | 16388 | 25078 | 33852 | 39646 | 41823 | 42412 | 43579 | 41203 |
| NB149-95 | 20451 | 21397 | 29400 | 37982 | 41364 | 42313 | 42277 | 42206 |
| Tab142 | 17127 | 23145 | 34784 | 40366 | 42730 | 42624 | 43729 | 42571 |
| hIgG1 | 41197 | 42563 | 43114 | 44294 | 43580 | 43690 | 43654 | |

TABLE 36
Results of Competition between VHH-hFc and Biotin-Tab142

| Competitive | Concentration (nM) | | | | | | | |
|---|---|---|---|---|---|---|---|---|
| Antibodies | 200 | 40 | 8 | 1.6 | 0.32 | 0.064 | 0.0128 | 0.00256 |
| NB149-97 | 115 | 265 | 845 | 13035 | 21328 | 23297 | 23598 | 23990 |
| Tab142 | 180 | 579 | 6272 | 19797 | 23360 | 23777 | 23506 | 23888 |
| hIgG1 | 23977 | 23629 | 23007 | 22698 | 23303 | 23610 | 24084 | 24269 |

TABLE 37
Results of Competition between VHH-hFc and Biotin-Tab131

| Concentra- | Competitive Antibodies | | | | | | | |
|---|---|---|---|---|---|---|---|---|
| tion (nM) | NB148-13 | NB148-25 | NB148-35 | NB149-88 | Tab020 | Tab142 | Tab131 | hIgG1 |
| 200 | 195 | 124 | 115 | 158 | 395 | 22504 | 2024 | 12866 |
| 40 | 582 | 977 | 1530 | 2054 | 5003 | 21532 | 6318 | 12519 |
| 8 | 7444 | 7570 | 8842 | 9390 | 10683 | 19510 | 11060 | 12990 |
| 1.6 | 11972 | 11614 | 11717 | 12196 | 12412 | 13551 | 12317 | 12633 |
| 0.32 | 11910 | 11690 | 12039 | 12593 | 12763 | 13838 | 12753 | 12670 |
| 0.064 | 12524 | 12490 | 12990 | 12578 | 13035 | 12777 | 12399 | 12468 |
| 0.0128 | 11793 | 11587 | 11887 | 11720 | 12123 | 12987 | 12652 | 12632 |
| 0.00256 | 12810 | 13302 | 13779 | 13888 | 13334 | 13218 | 13413 | |

1. An antibody or an antigen-binding fragment specifically binding to MSLN, comprising: a CDR1, a CDR2, and a CDR3, wherein amino acid sequences of the CDR1, the CDR2, and the CDR3 are:
(1) the CDR1, according to the KABAT, Chothia, or IMGT numbering scheme, respectively comprises a sequence set forth in SEQ ID NOs: 65, 101, and 137;
(2) the CDR2, according to the KABAT, Chothia, or IMGT numbering scheme, respectively comprises a sequence set forth in SEQ ID NOs: 66, 102, and 138;
(3) the CDR3, according to the KABAT, Chothia, or IMGT numbering scheme, respectively comprises a sequence set forth in SEQ ID NOs: 67, 103, and 139.

2. (canceled)

3. The antibody or the antigen-binding fragment according to claim 1, wherein the CDR1, the CDR2 and the CDR3 have a sequence combination with 1, 2, 3, or more amino acid insertions, deletions and/or substitutions, and preferably, the substitution is a conservative amino acid substitution.

4. The antibody or the antigen-binding fragment according to claim 1, wherein the said antibody or antigen-binding fragment comprises a sequence set forth in SEQ ID NO:33.

5. (canceled)

6. The antibody or the antigen-binding fragment according to claim 1, comprising a sequence set forth in SEQ ID NO: 33; optionally, the antibody or antigen-binding fragment comprises a sequence having at least 80%, 85%, 90%, 91%, 92%, 93%, 94%, 95%, 96%, 97%, 98%, 99%, or 100% sequence identity to the sequence set forth in SEQ ID NO: 33; or, optionally, the antibody or the antigen-binding fragment comprises a sequence having at most 20, 19, 18, 17, 16, 15, 14, 13, 12, 11, 10, 9, 8, 7, 6, 5, 4, 3, 2, or 1 mutation compared with the sequence set forth in SEQ ID NO: 33; the mutation is selected from an insertion, a deletion, and/or a substitution.

7. The antibody or the antigen-binding fragment according to claim 6, wherein the antibody or the antigen-binding fragment binds to human MSLN with a dissociation constant (KD) not greater than 20 nM.

8. The antibody or the antigen-binding fragment according to claim 1, wherein the antibody or the antigen-binding fragment comprises or does not comprise an antibody heavy chain constant region; optionally, the antibody heavy chain constant region is selected from human, Vicugna pacos, mouse, rat, rabbit, and sheep; optionally, the antibody heavy chain constant region is selected from IgG, IgM, IgA, IgE, and IgD, and the IgG is selected from IgG1, IgG2, IgG3, and IgG4; optionally, the heavy chain constant region is selected from an Fc region, a CH3 region, a heavy chain constant region without a CH1 fragment, and an intact heavy chain constant region.

9. The antibody or the antigen-binding fragment according to claim 1, wherein the antibody or the antigen-binding fragment is:
(1) a chimeric antibody or a fragment thereof;
(2) a humanized antibody or a fragment thereof; or
(3) a full human antibody or a fragment thereof.

10. The antibody or the antigen-binding according to claim 1, wherein the antibody or the antigen-binding fragment is further conjugated to a therapeutic agent or a tracer.

11. The antibody or the antigen-binding fragment according to claim 1, wherein the antibody or the antigen-binding fragment is further linked to an additional functional molecule; and wherein the additional functional molecule is selected from one or more of: a signal peptide, a protein tag, and a cytokine.

12. A multispecific antibody comprising the antibody or the antigen-binding fragment according to claim 1 and an antibody or an antigen-binding fragment that binds to an antigen other than MSLN or binds to an epitope of MSLN different from that of the antibody or the antigen-binding fragment according to claim 1.

13. The multispecific antibody according to claim 12, wherein the antigen other than MSLN is selected from: CD3; CD16; CD32B; PD-1; PD-2; PD-L1; VEGF; NKG2D; CD19; CD20; CD40; CD47; 4-1BB; CD137; EGFR; EGFRvIII; TNF-alpha; CD33; HER2; HER3; HAS; CD5; CD27; EphA2; EpCAM; MUC1; MUC16; CEA; Claudin18.2; folate receptor; Claudin6; WT1; NY-ESO-1; MAGE3; ASGPR1 and CDH16.

14. The multispecific antibody according to claim 12, wherein the multispecific antibody is a bispecific antibody, a trispecific antibody, or a tetraspecific antibody, and is bivalent, tetravalent, or hexavalent.

15. A chimeric antigen receptor (CAR) comprising an extracellular antigen-binding domain, a transmembrane domain, and an intracellular signaling domain, wherein the extracellular antigen-binding domain comprises the antibody or the antigen-binding fragment according to claim 1.

16. An immune effector cell expressing the CAR according to claim 15, or comprising a nucleic acid fragment encoding the CAR according to claim 15.

17. An isolated nucleic acid fragment encoding the antibody or the antigen-binding fragment according to claim 1.

18. (canceled)

19. (canceled)

20. (canceled)

21. A method for preparing an immune effector cell, the method comprising: introducing a nucleic acid fragment encoding the CAR according to claim 15 into the immune effector cell, and optionally initiating expression of the CAR in the immune effector cell.

22. A pharmaceutical composition comprising the antibody or the antigen-binding fragment according to claim 1, and optionally an additional antineoplastic agent.

23. (canceled)

24. A method for preventing and/or treating a tumor in a patient in need thereof, the method comprising: administering to the patient an effective amount of the antibody or the antigen-binding fragment according to claim 1.

25. (canceled)

26. (canceled)

27. (canceled)

28. (canceled)

29. The antibody or the antigen-binding fragment according to claim 8, wherein the antibody or the antigen-binding fragment is a single-domain antibody or a heavy-chain antibody.


SEQUENCE LISTING (142 sequences)

SEQ ID NO: 1 - protein, 122 aa, Artificial Sequence
  synthetic polypeptide
QEQLEESGGDLVQPEGSLTLTCKASGLDFSSSYWICWVRQAPGKGLEWIGCRHTFTANTWSASWVNGRFTISRSTSLGTVDLKMTSLTAADTATYFCARDESNNDGWDFKLWGPGTLVTVSS

SEQ ID NO: 2 - protein, 109 aa, Artificial Sequence
  synthetic polypeptide
AYDMTQTPASVSAAVGGTVTIKCQASQSISNYLAWYQQKPGQPPKLLIYQASTLAPGVSSRFKGSGSGTEFTLTISGVECADAATYYCQQGYTSSNVENVFGGGTGVVV

SEQ ID NO: 3 - protein, 123 aa, Artificial Sequence
  synthetic polypeptide
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSS

SEQ ID NO: 4 - protein, 110 aa, Artificial Sequence
  synthetic polypeptide
DVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 5 - protein, 119 aa, Artificial Sequence
  synthetic polypeptide
QEQLVESGGGLVQPGASLTLTCTASGIDFSRYYMCWVRQAPGKGLEGIACIYIGGSGSTYYASWAKGRFTISKASSTTVTLQMTSLTAADTATYFCARGTNLNYIFRLWGPGTLVTVSS

SEQ ID NO: 6 - protein, 109 aa, Artificial Sequence
  synthetic polypeptide
DVVMTQTPSPVSAAVGGTVTIKCQASQSINNGLAWYQQKPGQPPRLLIYSASNLESGVPSRFKGSGSGTEFTLTISDLECDDAATYYCQCIWDGNSYVNAFGGGTEVVV

SEQ ID NO: 7 - protein, 246 aa, Artificial Sequence
  synthetic polypeptide
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSSGGGGSGGGGSGGGGSRHQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKGQAG

SEQ ID NO: 8 - protein, 248 aa, Artificial Sequence
  synthetic polypeptide
QQQLEESGGGLVKPEGSLTLTCKASGFDLGFYFYACWVRQAPGKGLEWIACIYTAGSGSTYYASWAKGRFTISKASSTTVTLQMTSLAAADTATYFCARSTANTRSTYYLNLWGPGTLVTVSSGGGGSGGGGSGGGGSDVVMTQTPASVSEPVGGTVTIKCQASQRISSYLSWYQQKPGQRPKLLIFGASTLASGVPSRFKGSGSGTEYTLTISDLECADAATYYCQSYAYFDSNNWHAFGGGTEVVV

SEQ ID NO: 9 - protein, 449 aa, Artificial Sequence
  synthetic polypeptide
QVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, Artificial Sequence
  synthetic polypeptide
DIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 232 aa, Homo sapiens
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 107 aa, Homo sapiens [sequence omitted]

SEQ ID NO: 14 - protein, 323 aa, Oryctolagus cuniculus
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 15 - protein, 104 aa, Oryctolagus cuniculus
GDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 16 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAPEIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 17 - protein, 627 aa, Macaca fascicularis
MALPMARPLSGSCGTPAVGSLLFLLFSLGWVQPSRVLAGETRQALCPQEAAPLDGILTNAPDIASLSPRQLLGFTCVEVSGLSTELVQELAVALGQKNVKLSAEQLRCLAHRLSEPPEDLDALPLDLLLFLNPDAFSGPQACTHFFSRVAKANVDLLPRGAPERQRLLPAALTCWGVRGSLLSEADVRALGGLACDLPGCFVAESAEVVLPRLVRCLGPLDQDQQEAARAALQRGGPPYGPPSTWSISTLDDLQSLLPVLGQPVIHSIPKGILAAWRQRSSRDPSWQQPEQTVLRPRFRRDVERTTCPPEKEVHEIDESLIFYKKRELEACVDAALLAAQMDRVDAIPFTYEQLDVLKHKLDELYPQGYPESVIRHLGHLFLKMSPEDIRKWNVTSLETLKALLKVSKGHEMSAQVATLIDRVVVGRGQLDKDTVDTLTAFCPGCLCSLSPERLSSVPPSVIGAVRPQDLDTCGPRQLDVLYPKARLAFQNMSGSEYFVKIRPFLGGAPTEDVKALSQQNVSMDLATFMKLRREAVLPLTVAEVQKLLGPHVEGLKVEEQHSPVRDWILKQRQDDLDTLGLGLQGGIPNGYLILDLSVREALSGTPCLLGPGPVLTVLALLLASTLA

SEQ ID NO: 18 - DNA, 18 bp, Artificial Sequence
  synthetic polynucleotide
cttggtggtcctggctgc

SEQ ID NO: 19 - DNA, 23 bp, Artificial Sequence
  synthetic polynucleotide
ggtacgtgctgttgaactgttcc

SEQ ID NO: 20 - DNA, 47 bp, Artificial Sequence
  synthetic polynucleotide
catgccatgactgtggcccaggcggcccagktgcagctcgtggagtc

SEQ ID NO: 21 - DNA, 51 bp, Artificial Sequence
  synthetic polynucleotide
catgccatgactcgcggccggcctggccatgggggtcttcgctgtggtgcg

SEQ ID NO: 22 - DNA, 52 bp, Artificial Sequence
  synthetic polynucleotide
catgccatgactcgcggccggcctggccgtcttgtggttttggtgtcttggg

SEQ ID NO: 23 - protein, 111 aa, Artificial Sequence
  synthetic polypeptide
QLQLVESGGGLVQAGGSLRLSCTASGSTSMSYGAKWFRRAPGKEREWVASVYRDGTTGYDDSVKGRFTISRDLAKNTVYLQMNSLRLEDTAVYYCTALNSWGQGTQVTVSS

SEQ ID NO: 24 - protein, 115 aa, Artificial Sequence
  synthetic polypeptide
QVQLVESGGGLVQAGGSLRLSCAASGSTFGRIVMRWYRQAQGKQREWVASIYDSGSTSYSDPVQGRFTISRDNARNTVYLQMNSLRPADTAVYYCNLGTALSSYWGQGTQVTVSS

SEQ ID NO: 25 - protein, 127 aa, Artificial Sequence
  synthetic polypeptide
EVQVVESGGGLVQAGASLRLSCASSGRTISPIAMAWFRQAPGKERELVAGLTWTGRTYYSDSVKGRFTISRDNAKNTVYLQMDSLKFEDTAVYFCAAGDNPDRRGSSWSSSSVYEYWGQGTQVTVSS

SEQ ID NO: 26 - protein, 116 aa, Artificial Sequence
  synthetic polypeptide
EVQVVESGGGLVQPGGSLRLSCGVSGSILALNTMRWYRQTPGNQREWVASIYSSGGSTGYADSVKGRFIISRDSAKNMMSLQMNSLKSEDTAVYYCNVQTPLNVYWGQGTQVTVSS

SEQ ID NO: 27 - protein, 117 aa, Artificial Sequence
  synthetic polypeptide
EVQLVESGGGLVQAGGSLRLSCAASGSTFSSYPMGWYRQAPGKQRELVAAISSGGSTNYADSVKGRFTISRDNAKNTVYLQMNSLKPEDTAVYYCNTRSPRVPNDSWGQGTQVTVSS

SEQ ID NO: 28 - protein, 115 aa, Artificial Sequence
  synthetic polypeptide
QLQLVESGGGLVQPGGSHRLSCAVSVSTFTRYAMRWYRQAPGKEREWLTSIYSDGSSAYADSVKDRFTISLDNAKNMLYLQMNSLKPEDTAVYYCNANVDSKAYWGQGTQVTVAS

SEQ ID NO: 29 - protein, 121 aa, Artificial Sequence
  synthetic polypeptide
EVQVVESGGGLVQPGGSLRLSCAASGITFSGNYGGWFRQAPGEQRELVASMSSALGTKYADPVKGRFTISTDTAKNTVDLQMNSLGPEDTAVYYCYARVRLSRGGWGSEYWGQGTQVTVSS

SEQ ID NO: 30 - protein, 119 aa, Artificial Sequence
  synthetic polypeptide
QLQLVESGGGLVQPGGSLRLSCAASGSVNSITAMGWYRQGPGNERELVAVITDRGRASYADSVKGRFAISMDNDKHTVYLQMDSLKPEDTAVYYCNAHRRTWPHPGDYWGQGTQVTVSS

SEQ ID NO: 31 - protein, 116 aa, Artificial Sequence
  synthetic polypeptide
QVQLVESGGGLVQPGGSLRLSCAASGSIFSGNAVAWYRQAPGKEREAVAVITRDGDTKYADSVKGRFTISRYNAKNIAYLQMNSLKPEDTAVYYCNTARGAAVDSWGQGTQVTVSS

SEQ ID NO: 32 - protein, 122 aa, Artificial Sequence
  synthetic polypeptide
EVQLVESGGGLVQAGGALRLSCAASERTFSSSSMGWFRQAPGKEREFVAAINRIGGHIYYADSVKGRFTISKDNAKNTVYLQMSSLKPEDTAVYYCASSRIYSHSARDYDYWGQGTQVTVSS

SEQ ID NO: 33 - protein, 116 aa, Artificial Sequence
  synthetic polypeptide
QVQLVESGGGLVQAGGSLRLSCTASESGFSANYMGWYRQEAPGKERELVATINRFGSTNYADSVKGRFTISRDNAKNTVYLQMNSLKSEDTGVYYCRIMRPGNWYWGQGTQVTVSS

SEQ ID NO: 34 - protein, 122 aa, Artificial Sequence
  synthetic polypeptide
EVQVVESGGGLVQAGGGLRLSCAASERTFSSSSIGWFRQAPGKEREFVATMNRIGGHTFYADPVKGRFTISRDNAANTVYLQMSSLKPEDAAVYYCASSRIYSHSPRDYDYWSQGTQVTVSS

SEQ ID NO: 35 - protein, 5 aa, Artificial Sequence
  synthetic polypeptide
SYGAK

SEQ ID NO: 36 - protein, 16 aa, Artificial Sequence
  synthetic polypeptide
SVYRDGTTGYDDSVKG

SEQ ID NO: 37 - skipped

SEQ ID NO: 38 - protein, 5 aa, Artificial Sequence
  synthetic polypeptide
RIVMR

SEQ ID NO: 39 - protein, 16 aa, Artificial Sequence
  synthetic polypeptide
SIYDSGSTSYSDPVQG

SEQ ID NO: 40 - protein, 7 aa, Artificial Sequence
  synthetic polypeptide
GTALSSY

SEQ ID NO: 41 - protein, 5 aa, Artificial Sequence
  synthetic polypeptide
PIAMA

SEQ ID NO: 42 - protein, 16 aa, Artificial Sequence
  synthetic polypeptide
GLTWTGRTYYSDSVKG

SEQ ID NO: 43 - protein, 19 aa, Artificial Sequence
  synthetic polypeptide
GDNPDRRGSSWSSSSVYEY

SEQ ID NO: 44 - protein, 5 aa, Artificial Sequence
  synthetic polypeptide
LNTMR

SEQ ID NO: 45 - protein, 17 aa, Artificial Sequence
  synthetic polypeptide
SIYSSGGSTGYADSVKG

SEQ ID NO: 46 - protein, 7 aa, Artificial Sequence
  synthetic polypeptide
QTPLNVY

SEQ ID NO: 47 - protein, 5 aa, Artificial Sequence
  synthetic polypeptide
SYPMG

SEQ ID NO: 48 - protein, 16 aa, Artificial Sequence
  synthetic polypeptide
AISSGGSTNYADSVKG

SEQ ID NO: 49 - protein, 9 aa, Artificial Sequence
  synthetic polypeptide
RSPRVPNDS

SEQ ID NO: 50 - protein, 5 aa, Artificial Sequence
  synthetic polypeptide
RYAMR

SEQ ID NO: 51 - protein, 16 aa, Artificial Sequence
  synthetic polypeptide
SIYSDGSSAYADSVKD

SEQ ID NO: 52 - protein, 7 aa, Artificial Sequence
  synthetic polypeptide
NVDSKAY

SEQ ID NO: 53 - protein, 5 aa, Artificial Sequence
  synthetic polypeptide
GNYGG

SEQ ID NO: 54 - protein, 16 aa, Artificial Sequence
  synthetic polypeptide
SMSSALGTKYADPVKG

SEQ ID NO: 55 - protein, 13 aa, Artificial Sequence
  synthetic polypeptide
RVRLSRGGWGSEY

SEQ ID NO: 56 - protein, 5 aa, Artificial Sequence
  synthetic polypeptide
ITAMG

SEQ ID NO: 57 - protein, 16 aa, Artificial Sequence
  synthetic polypeptide
VITDRGRASYADSVKG

SEQ ID NO: 58 - protein, 11 aa, Artificial Sequence
  synthetic polypeptide
HRRTWPHPGDY

SEQ ID NO: 59 - protein, 5 aa, Artificial Sequence
  synthetic polypeptide
GNAVA

SEQ ID NO: 60 - protein, 16 aa, Artificial Sequence
  synthetic polypeptide
VITRDGDTKYADSVKG

SEQ ID NO: 61 - protein, 8 aa, Artificial Sequence
  synthetic polypeptide
ARGAAVDS

SEQ ID NO: 62 - protein, 5 aa, Artificial Sequence
  synthetic polypeptide
SSSMG

SEQ ID NO: 63 - protein, 17 aa, Artificial Sequence
  synthetic polypeptide
AINRIGGHIYYADSVKG

SEQ ID NO: 64 - protein, 13 aa, Artificial Sequence
  synthetic polypeptide
SRIYSHSARDYDY

SEQ ID NO: 65 - protein, 5 aa, Artificial Sequence
  synthetic polypeptide
ANYMG

SEQ ID NO: 66 - protein, 16 aa, Artificial Sequence
  synthetic polypeptide
TINRFGSTNYADSVKG

SEQ ID NO: 67 - protein, 7 aa, Artificial Sequence
  synthetic polypeptide
MRPGNWY

SEQ ID NO: 68 - protein, 5 aa, Artificial Sequence
  synthetic polypeptide
SSSIG

SEQ ID NO: 69 - protein, 17 aa, Artificial Sequence
  synthetic polypeptide
TMNRIGGHTFYADPVKG

SEQ ID NO: 70 - protein, 13 aa, Artificial Sequence
  synthetic polypeptide
SRIYSHSPRDYDY

SEQ ID NO: 71 - protein, 7 aa, Artificial Sequence
  synthetic polypeptide
GSTSMSY

SEQ ID NO: 72 - protein, 5 aa, Artificial Sequence
  synthetic polypeptide
YRDGT

SEQ ID NO: 73 - skipped

SEQ ID NO: 74 - protein, 7 aa, Artificial Sequence
  synthetic polypeptide
GSTFGRI

SEQ ID NO: 75 - protein, 5 aa, Artificial Sequence
  synthetic polypeptide
YDSGS

SEQ ID NO: 76 - protein, 7 aa, Artificial Sequence
  synthetic polypeptide
GTALSSY

SEQ ID NO: 77 - protein, 7 aa, Artificial Sequence
  synthetic polypeptide
GRTISPI

SEQ ID NO: 78 - protein, 5 aa, Artificial Sequence
  synthetic polypeptide
TWTGR

SEQ ID NO: 79 - protein, 19 aa, Artificial Sequence
  synthetic polypeptide
GDNPDRRGSSWSSSSVYEY

SEQ ID NO: 80 - protein, 7 aa, Artificial Sequence
  synthetic polypeptide
GSILALN

SEQ ID NO: 81 - protein, 6 aa, Artificial Sequence
  synthetic polypeptide
YSSGGS

SEQ ID NO: 82 - protein, 7 aa, Artificial Sequence
  synthetic polypeptide
QTPLNVY

SEQ ID NO: 83 - protein, 7 aa, Artificial Sequence
  synthetic polypeptide
GSTFSSY

SEQ ID NO: 84 - protein, 5 aa, Artificial Sequence
  synthetic polypeptide
SSGGS

SEQ ID NO: 85 - protein, 9 aa, Artificial Sequence
  synthetic polypeptide
RSPRVPNDS

SEQ ID NO: 86 - protein, 7 aa, Artificial Sequence
  synthetic polypeptide
VSTFTRY

SEQ ID NO: 87 - protein, 5 aa, Artificial Sequence
  synthetic polypeptide
YSDGS

SEQ ID NO: 88 - protein, 7 aa, Artificial Sequence
  synthetic polypeptide
NVDSKAY

SEQ ID NO: 89 - protein, 7 aa, Artificial Sequence
  synthetic polypeptide
GITFSGN

SEQ ID NO: 90 - protein, 5 aa, Artificial Sequence
  synthetic polypeptide
SSALG

SEQ ID NO: 91 - protein, 13 aa, Artificial Sequence
  synthetic polypeptide
RVRLSRGGWGSEY

SEQ ID NO: 92 - protein, 7 aa, Artificial Sequence
  synthetic polypeptide
GSVNSIT

SEQ ID NO: 93 - protein, 5 aa, Artificial Sequence
  synthetic polypeptide
TDRGR

SEQ ID NO: 94 - protein, 11 aa, Artificial Sequence
  synthetic polypeptide
HRRTWPHPGDY

SEQ ID NO: 95 - protein, 7 aa, Artificial Sequence
  synthetic polypeptide
GSIFSGN

SEQ ID NO: 96 - protein, 5 aa, Artificial Sequence
  synthetic polypeptide
TRDGD

SEQ ID NO: 97 - protein, 8 aa, Artificial Sequence
  synthetic polypeptide
ARGAAVDS

SEQ ID NO: 98 - protein, 7 aa, Artificial Sequence
  synthetic polypeptide
ERTFSSS

SEQ ID NO: 99 - protein, 6 aa, Artificial Sequence
  synthetic polypeptide
NRIGGH

SEQ ID NO: 100 - protein, 13 aa, Artificial Sequence
  synthetic polypeptide
SRIYSHSARDYDY

SEQ ID NO: 101 - protein, 7 aa, Artificial Sequence
  synthetic polypeptide
ESGFSAN

SEQ ID NO: 102 - protein, 5 aa, Artificial Sequence
  synthetic polypeptide
NRFGS

SEQ ID NO: 103 - protein, 7 aa, Artificial Sequence
  synthetic polypeptide
MRPGNWY

SEQ ID NO: 104 - protein, 7 aa, Artificial Sequence
  synthetic polypeptide
ERTFSSS

SEQ ID NO: 105 - protein, 6 aa, Artificial Sequence
  synthetic polypeptide
NRIGGH

SEQ ID NO: 106 - protein, 13 aa, Artificial Sequence
  synthetic polypeptide
SRIYSHSPRDYDY

SEQ ID NO: 107 - protein, 8 aa, Artificial Sequence
  synthetic polypeptide
GSTSMSYG

SEQ ID NO: 108 - protein, 7 aa, Artificial Sequence
  synthetic polypeptide
VYRDGTT

SEQ ID NO: 109 - protein, 5 aa, Artificial Sequence
  synthetic polypeptide
TALNS

SEQ ID NO: 110 - protein, 8 aa, Artificial Sequence
  synthetic polypeptide
GSTFGRIV

SEQ ID NO: 111 - protein, 7 aa, Artificial Sequence
  synthetic polypeptide
IYDSGST

SEQ ID NO: 112 - protein, 9 aa, Artificial Sequence
  synthetic polypeptide
NLGTALSSY

SEQ ID NO: 113 - protein, 8 aa, Artificial Sequence
  synthetic polypeptide
GRTISPIA

SEQ ID NO: 114 - protein, 7 aa, Artificial Sequence
  synthetic polypeptide
LTWTGRT

SEQ ID NO: 115 - protein, 21 aa, Artificial Sequence
  synthetic polypeptide
AAGDNPDRRGSSWSSSSVYEY

SEQ ID NO: 116 - protein, 8 aa, Artificial Sequence
  synthetic polypeptide
GSILALNT

SEQ ID NO: 117 - protein, 8 aa, Artificial Sequence
  synthetic polypeptide
IYSSGGST

SEQ ID NO: 118 - protein, 9 aa, Artificial Sequence
  synthetic polypeptide
NVQTPLNVY

SEQ ID NO: 119 - protein, 8 aa, Artificial Sequence
  synthetic polypeptide
GSTFSSYP

SEQ ID NO: 120 - protein, 7 aa, Artificial Sequence
  synthetic polypeptide
ISSGGST

SEQ ID NO: 121 - protein, 11 aa, Artificial Sequence
  synthetic polypeptide
NTRSPRVPNDS

SEQ ID NO: 122 - protein, 8 aa, Artificial Sequence
  synthetic polypeptide
VSTFTRYA

SEQ ID NO: 123 - protein, 7 aa, Artificial Sequence
  synthetic polypeptide
IYSDGSS

SEQ ID NO: 124 - protein, 9 aa, Artificial Sequence
  synthetic polypeptide
NANVDSKAY

SEQ ID NO: 125 - protein, 8 aa, Artificial Sequence
  synthetic polypeptide
GITFSGNY

SEQ ID NO: 126 - protein, 7 aa, Artificial Sequence
  synthetic polypeptide
MSSALGT

SEQ ID NO: 127 - protein, 15 aa, Artificial Sequence
  synthetic polypeptide
YARVRLSRGGWGSEY

SEQ ID NO: 128 - protein, 8 aa, Artificial Sequence
  synthetic polypeptide
GSVNSITA

SEQ ID NO: 129 - protein, 7 aa, Artificial Sequence
  synthetic polypeptide
ITDRGRA

SEQ ID NO: 130 - protein, 13 aa, Artificial Sequence
  synthetic polypeptide
NAHRRTWPHPGDY

SEQ ID NO: 131 - protein, 8 aa, Artificial Sequence
  synthetic polypeptide
GSIFSGNA

SEQ ID NO: 132 - protein, 7 aa, Artificial Sequence
  synthetic polypeptide
ITRDGDT

SEQ ID NO: 133 - protein, 10 aa, Artificial Sequence
  synthetic polypeptide
NTARGAAVDS

SEQ ID NO: 134 - protein, 8 aa, Artificial Sequence
  synthetic polypeptide
ERTFSSSS

SEQ ID NO: 135 - protein, 8 aa, Artificial Sequence
  synthetic polypeptide
INRIGGHI

SEQ ID NO: 136 - protein, 15 aa, Artificial Sequence
  synthetic polypeptide
ASSRIYSHSARDYDY

SEQ ID NO: 137 - protein, 8 aa, Artificial Sequence
  synthetic polypeptide
ESGFSANY

SEQ ID NO: 138 - protein, 7 aa, Artificial Sequence
  synthetic polypeptide
INRFGST

SEQ ID NO: 139 - protein, 9 aa, Artificial Sequence
  synthetic polypeptide
RIMRPGNWY

SEQ ID NO: 140 - protein, 8 aa, Artificial Sequence
  synthetic polypeptide
ERTFSSSS

SEQ ID NO: 141 - protein, 8 aa, Artificial Sequence
  synthetic polypeptide
MNRIGGHT

SEQ ID NO: 142 - protein, 15 aa, Artificial Sequence
  synthetic polypeptide
ASSRIYSHSPRDYDY